(19) United States
(12) Patent Application Publication
(10) Pub. No.: US 2022/0005859 A1
(43) Pub. Date: Jan. 6, 2022

(54) SOLID-STATE IMAGING DEVICE AND ELECTRONIC APPARATUS

(71) Applicant: SONY SEMICONDUCTOR SOLUTIONS CORPORATION, KANAGAWA (JP)

(72) Inventor: KEISUKE HATANO, KANAGAWA (JP)

(21) Appl. No.: 17/291,100

(22) PCT Filed: Nov. 7, 2019
(86) PCT No.: PCT/JP2019/043665

§ 371 Date: May 4, 2021

(30) Foreign Application Priority Data
Nov. 15, 2018 (JP) 2018-214522

(51) Int. Cl. H01L 27/146

(52) U.S. Cl. CPC H01L 27/14636; A61B 1/05; H01L 27/1461; H01L 27/14645

(57) ABSTRACT

The present disclosure is to provide a solid-state imaging device capable of further increasing the degree of freedom in the design of rewiring lines connecting to joining portions that are contact points between a solid-state imaging device and a module substrate on which the solid-state imaging device is mounted.

The present technology provides a solid-state imaging device that includes: a sensor substrate that includes a first semiconductor substrate having a first principal surface on a light incident side and a second principal surface on a side opposite from the first principal surface, a light receiving element being disposed two-dimensionally on the first principal surface, and a first wiring layer formed on the second principal surface of the first semiconductor substrate; a circuit substrate that includes a second semiconductor substrate having a third principal surface on the light incident side and a fourth principal surface on a side opposite from the third principal surface, and a second wiring layer formed on the third principal surface of the second semiconductor substrate; a light transmissive substrate disposed above the light receiving element; a first rewiring line electrically connected to an internal electrode formed in the second wiring layer; and a second rewiring line formed on a side of the fourth principal surface of the second semiconductor substrate. In the solid-state imaging device, the first wiring layer of the sensor substrate and the second wiring layer of the circuit substrate are bonded, to form a stack structure of the sensor substrate and the circuit substrate.

### TECHNICAL FIELD

The present technology relates to solid-state imaging devices and electronic apparatuses.

### BACKGROUND ART

In a camera module to be used in a smartphone or the like, the camera module is required to be smaller and thinner to make the smartphone smaller and thinner.

For example, a technology has been suggested for miniaturizing a solid-state imaging device using a packaging technology with a chip-sized package (see Patent Document 1).

### CITATION LIST

### Patent Document

Patent Document 1: Japanese Patent Application Laid-Open No. 2015-135938

### SUMMARY OF THE INVENTION

### Problems to be Solved by the Invention

By the technology suggested in Patent Document 1, however, there is a possibility that it will not be possible to further increase the degree of freedom in designing rewiring lines connecting to joining portions (such as connection terminals, for example) that are the contact points between a solid-state imaging device and a module substrate on which the solid-state imaging device is mounted.

Therefore, the present technology has been made in view of such circumstances, and the principal object thereof is to provide a solid-state imaging device capable of further increasing the degree of freedom in rewiring design, and an electronic apparatus equipped with the solid-state imaging device.

### Solutions to Problems

As a result of intensive studies conducted to achieve the above object, the present inventor has succeeded in further increasing the degree of freedom in the design of rewiring lines included in a solid-state imaging device, and has completed the present technology.

Specifically, the present technology provides, as a first aspect,

a solid-state imaging device that includes:

a semiconductor substrate that has a first principal surface on the light incident side and a second principal surface on the side opposite from the first principal surface, a light receiving element being disposed two-dimensionally on the first principal surface;

a light transmissive substrate disposed above the light receiving element;

a wiring layer formed on the second principal surface of the semiconductor substrate;

a first rewiring line electrically connected to an internal electrode formed in the wiring layer; and

a second rewiring line formed on the side of the second principal surface of the semiconductor substrate.

In the solid-state imaging device of the first aspect according to the present technology,

the second rewiring line may be disposed below the first rewiring line.

In the solid-state imaging device of the first aspect according to the present technology,

the first rewiring line may be formed with a plurality of first rewiring lines,

the second rewiring line may be formed with a plurality of second rewiring lines, and

at least one first rewiring line of the plurality of first rewiring lines and at least one second rewiring line of the plurality of second rewiring lines may be connected.

In the solid-state imaging device of the first aspect according to the present technology,

the first rewiring line may be formed with a plurality of first rewiring lines,

the second rewiring line may be formed with a plurality of second rewiring lines,

the wiring layer may include a plurality of signal lines formed in a predetermined direction, and

the plurality of first rewiring lines and the plurality of second rewiring lines may be arranged to generate a plurality of magnetic fields having different magnetic directions from one another in a region between two adjacent signal lines of the plurality of signal lines.

In the solid-state imaging device of the first aspect according to the present technology,

at least one pair of one first rewiring line of the plurality of first rewiring lines and one second rewiring line of the plurality of second rewiring lines may be formed in a vertical direction, and,

in the one pair, the direction of a first current flowing in the one first rewiring line and the direction of a second current flowing in the one second rewiring line may be the opposite from each other.

In the solid-state imaging device of the first aspect according to the present technology,

the first rewiring line may be formed with a plurality of first rewiring lines,

the second rewiring line may be formed with a plurality of second rewiring lines,

the wiring layer may include a plurality of signal lines formed in a predetermined direction, and

at least one first rewiring line of the plurality of first rewiring lines and/or at least one second rewiring line of the plurality of second rewiring lines may cover at least part of at least one signal line of the plurality of signal lines, when viewed from the side opposite from the light incident side.

In the solid-state imaging device of the first aspect according to the present technology,

the first rewiring line may be formed with a plurality of first rewiring lines,

a groove portion may be formed on the side of the second principal surface of the semiconductor substrate, and

at least part of at least one first rewiring line of the plurality of first rewiring lines may be formed in the groove portion.

In the solid-state imaging device of the first aspect according to the present technology,

the depth of the groove portion may be equal to or greater than the thickness of at least one first rewiring line of the plurality of first rewiring lines.

The present technology also provides, as a second aspect,

a solid-state imaging device that includes:

a sensor substrate that includes:

a first semiconductor substrate having a first principal surface on the light incident side and a second principal surface on the side opposite from the first principal surface, a light receiving element being disposed two-dimensionally on the first principal surface; and

a first wiring layer formed on the second principal surface of the first semiconductor substrate;

a circuit substrate that includes:

a second semiconductor substrate having a third principal surface on the light incident side and a fourth principal surface on the side opposite from the third principal surface; and

a second wiring layer formed on the third principal surface of the second semiconductor substrate;

a light transmissive substrate disposed above the light receiving element;

a first rewiring line electrically connected to an internal electrode formed in the second wiring layer; and

a second rewiring line formed on the side of the fourth principal surface of the second semiconductor substrate,

in which the first wiring layer of the sensor substrate and the second wiring layer of the circuit substrate are bonded, to form a stack structure of the sensor substrate and the circuit substrate.

In the solid-state imaging device of the second aspect according to the present technology,

the second rewiring line may be disposed below the first rewiring line.

In the solid-state imaging device of the second aspect according to the present technology,

the first rewiring line may be formed with a plurality of first rewiring lines,

the second rewiring line may be formed with a plurality of second rewiring lines, and

at least one first rewiring line of the plurality of first rewiring lines and at least one second rewiring line of the plurality of second rewiring lines may be connected.

In the solid-state imaging device of the second aspect according to the present technology,

the first rewiring line may be formed with a plurality of first rewiring lines,

the second rewiring line may be formed with a plurality of second rewiring lines,

the second wiring layer may include a plurality of signal lines formed in a predetermined direction, and

the plurality of first rewiring lines and the plurality of second wiring layers may be arranged to generate a plurality of magnetic fields having different magnetic directions from one another in a region between two adjacent signal lines of the plurality of signal lines.

In the solid-state imaging device of the second aspect according to the present technology,

at least one pair of one first rewiring line of the plurality of first rewiring lines and one second rewiring line of the plurality of second rewiring lines may be formed in a vertical direction, and,

in the one pair, the direction of a first current flowing in the one first rewiring line and the direction of a second current flowing in the one second rewiring line may be the opposite from each other.

In the solid-state imaging device of the second aspect according to the present technology,

the first rewiring line may be formed with a plurality of first rewiring lines,

the second rewiring line may be formed with a plurality of second rewiring lines,

the wiring layer may include a plurality of signal lines formed in a predetermined direction, and

at least one first rewiring line of the plurality of first rewiring lines and/or at least one second rewiring line of the plurality of second rewiring lines may cover at least part of at least one signal line of the plurality of signal lines, when viewed from the side opposite from the light incident side.

In the solid-state imaging device of the second aspect according to the present technology,

the first rewiring line may be formed with a plurality of first rewiring lines,

a groove portion may be formed on the side of the fourth principal surface of the second semiconductor substrate, and

at least part of at least one first rewiring line of the plurality of first rewiring lines may be formed in the groove portion.

In the solid-state imaging device of the second aspect according to the present technology,

the depth of the groove portion may be equal to or greater than the thickness of at least one first rewiring line of the plurality of first rewiring lines.

The present technology further provides an electronic apparatus on which the solid-state imaging device of the first aspect according to the present technology or the solid-state imaging device of the second aspect according to the present technology is mounted.

### Effects of the Invention

According to the present technology, it is possible to further increase the degree of freedom in designing the rewiring lines included in a solid-state imaging device. Note that effects of the present technology are not limited to the effects described herein, and may include any of the effects described in the present disclosure.

### BRIEF DESCRIPTION OF DRAWINGS

FIG. 1 is a cross-sectional diagram showing an example configuration of a solid-state imaging device to which the present technology is applied.

FIG. 2 is a cross-sectional diagram for explaining a method for manufacturing a solid-state imaging device to which the present technology is applied.

FIG. 3 is a cross-sectional diagram for explaining a method for manufacturing a solid-state imaging device to which the present technology is applied.

FIG. 4 is a cross-sectional diagram for explaining a method for manufacturing a solid-state imaging device to which the present technology is applied.

FIG. 5 is a cross-sectional diagram for explaining a method for manufacturing a solid-state imaging device to which the present technology is applied.

FIG. 6 is a cross-sectional diagram for explaining a method for manufacturing a solid-state imaging device to which the present technology is applied.

FIG. 7 is a cross-sectional diagram for explaining a method for manufacturing a solid-state imaging device to which the present technology is applied.

FIG. 8 is a cross-sectional diagram for explaining a method for manufacturing a solid-state imaging device to which the present technology is applied.

FIG. 9 is a cross-sectional diagram for explaining a method for manufacturing a solid-state imaging device to which the present technology is applied.

FIG. 10 is a diagram showing an example configuration of a solid-state imaging device to which the present technology is applied.

FIG. 11 is a plan view of an example configuration of a plurality of first rewiring lines included in a solid-state imaging device to which the present technology is applied.

FIG. 12 is a plan view of an example configuration of a plurality of second rewiring lines included in a solid-state imaging device to which the present technology is applied.

FIG. 13 is a cross-sectional diagram showing an example configuration of a plurality of first rewiring lines and an example configuration of a plurality of second rewiring lines included in a solid-state imaging device to which the present technology is applied.

FIG. 14 is a perspective view of an example configuration of a plurality of first rewiring lines, a plurality of second rewiring lines, and a plurality of signal lines included in a solid-state imaging device to which the present technology is applied.

FIG. 15 is a cross-sectional diagram showing an example configuration of a solid-state imaging device to which the present technology is applied.

FIG. 16 is a cross-sectional diagram showing an example configuration of a solid-state imaging device to which the present technology is applied.

FIG. 17 is a cross-sectional diagram for explaining a method for manufacturing a solid-state imaging device to which the present technology is applied.

FIG. 18 is a cross-sectional diagram for explaining a method for manufacturing a solid-state imaging device to which the present technology is applied.

FIG. 19 is a cross-sectional diagram for explaining a method for manufacturing a solid-state imaging device to which the present technology is applied.

FIG. 20 is a cross-sectional diagram for explaining a method for manufacturing a solid-state imaging device to which the present technology is applied.

FIG. 21 is a cross-sectional diagram for explaining a method for manufacturing a solid-state imaging device to which the present technology is applied.

FIG. 22 is a cross-sectional diagram for explaining a method for manufacturing a solid-state imaging device to which the present technology is applied.

FIG. 23 is a cross-sectional diagram for explaining a method for manufacturing a solid-state imaging device to which the present technology is applied.

FIG. 24 is a cross-sectional diagram for explaining a method for manufacturing a solid-state imaging device to which the present technology is applied.

FIG. 25 is a cross-sectional diagram for explaining a method for manufacturing a solid-state imaging device to which the present technology is applied.

FIG. 26 is a cross-sectional diagram showing an example configuration of a solid-state imaging device to which the present technology is applied.

FIG. 27 is a diagram showing examples of use of solid-state imaging devices of first to fifth embodiments to which the present technology is applied.

FIG. 28 is a functional block diagram of an example of an electronic apparatus according to a sixth embodiment to which the present technology is applied.

FIG. 29 is a diagram schematically showing an example configuration of an endoscopic surgery system.

FIG. 30 is a block diagram showing an example of the functional configurations of a camera head and a CCU.

FIG. 31 is a block diagram schematically showing an example configuration of a vehicle control system.

FIG. 32 is an explanatory diagram showing an example of installation positions of external information detectors and imaging units.

### MODES FOR CARRYING OUT THE INVENTION

The following is a description of preferred embodiments for carrying out the present technology. The embodiments described below are typical examples of embodiments of the present technology, and do not narrow the interpretation of the scope of the present technology. Note that “upper” means an upward direction or the upper side in the drawings, “lower” means a downward direction or the lower side in the drawings, “left” means a leftward direction or the left side in the drawings, and “right” means a rightward direction or the right side in the drawings, unless otherwise specified. Also, in the drawings, the same or equivalent components or members are denoted by the same reference numerals, and explanation of them will not be repeated.

Explanation will be made in the following order.

1. Outline of the present technology

2. First embodiment (Example 1 of a solid-state imaging device)

3. Second embodiment (Example 2 of a solid-state imaging device)

4. Third embodiment (Example 3 of a solid-state imaging device)

5. Fourth embodiment (Example 4 of a solid-state imaging device)

6. Fifth embodiment (Example 5 of a solid-state imaging device)

7. Sixth embodiment (an example of an electronic apparatus)

8. Examples of use of solid-state imaging devices to which the present technology is applied

9. Example application to an endoscopic surgery system

10. Example applications to mobile structures

### 1. Outline of the Present Technology

First, the outline of the present technology is described.

A solid-state imaging device of a chip-sized package type is formed by stacking a sensor substrate (also called an image sensor substrate, which will apply in the description below) in which a pixel region is formed, and a circuit substrate (a logic circuit substrate, for example) that processes pixel signals output from the pixel unit, for example. Photoelectric conversion units (photodiodes (PDs), for example) are formed in the semiconductor substrate included in the sensor substrate, and a light transmissive substrate is formed, via an adhesive layer, over on-chip lenses disposed above the semiconductor substrate included in the sensor substrate. Further, rewiring lines are provided under the circuit substrate (on the opposite side from the light incident side), so that terminals connecting to the module substrate are pulled out. The rewiring lines, which are the wiring lines connecting to the connection terminals herein, are formed with a single-layer metallic film, for example, and the degree of freedom in designing the rewiring lines might be low.

There also is a technology for forming rewiring lines having a two-layer configuration, for example. This technology is not used in solid-state imaging devices. Since this technology is designed to pull out the terminals for module connection from the principal surface side of the device, this technology differs in the thickness order of rewiring lines from the technology for a solid-state imaging device having rewiring lines in through holes formed in a silicon substrate (a semiconductor substrate) of a thickness on the order of a hundred microns. In some cases, to apply this technology to a solid-state imaging device, it is necessary to make great changes to the interlayer film structure between rewiring lines to be stacked, and the manufacturing method.

Further, in a solid-state imaging device, noise may be added to an output signal from a pixel due to the structure and the layout of rewiring lines. Therefore, in some cases, it is necessary to form rewiring lines by taking into account the influence on the imaging characteristics.

The present technology has been developed in view of the above circumstances. According to the present technology, it is possible to increase the degree of freedom in the rewiring line layout or improve imaging characteristics in a solid-state imaging device of a chip-sized package type, and further, both an increase in the degree of freedom in the rewiring line layout and improvement of imaging characteristics can be achieved.

Further, in a solid-state imaging device according to the present technology, the wiring lines connecting to the connection terminals are formed with a plurality of layers of rewiring lines formed on a substrate on the opposite side from the surface on the light incident side having a light transmissive substrate disposed thereon. With this arrangement, the degree of freedom in designing the rewiring lines can be increased, and furthermore, image noise can be reduced by the layout of the rewiring lines in the stack structure.

In the description below, embodiments according to the present technology will be explained in detail.

### 2. First Embodiment (Example 1 of a Solid-State Imaging Device)

A solid-state imaging device of a first embodiment (Example 1 of a solid-state imaging device) according to the present technology includes: a sensor substrate that includes a first semiconductor substrate having a first principal surface on the light incident side and a second principal surface on the side opposite from the first principal surface, light receiving elements being formed and arranged two-dimensionally on the first principal surface, and a first wiring layer formed on the second principal surface of the first semiconductor substrate; a circuit substrate that includes a second semiconductor substrate having a third principal surface on the light incident side and a fourth principal surface on the side opposite from the third principal surface, and a second wiring layer formed on the third principal surface of the second semiconductor substrate; a light transmissive substrate disposed above the light receiving elements; a first rewiring line electrically connected to an internal electrode formed in the second wiring layer; and a second rewiring line formed on the side of the fourth principal surface of the second semiconductor substrate. In this solid-state imaging device, the first wiring layer of the sensor substrate and the second wiring layer of the circuit substrate are bonded, to form a stack structure of the sensor substrate and the circuit substrate.

In the solid-state imaging device of the first embodiment (Example 1 of a solid-state imaging device) according to the present technology, the second rewiring line may be disposed below the first rewiring line. Also, in the solid-state imaging device of the first embodiment (Example 1 of a solid-state imaging device) according to the present technology, the first rewiring line may be formed with a plurality of first rewiring lines, the second rewiring line may be formed with a plurality of second rewiring lines, and at least one first rewiring line of the plurality of first rewiring lines and at least one second rewiring line of the plurality of second rewiring lines may be connected.

With the solid-state imaging device of the first embodiment (Example 1 of a solid-state imaging device) according to the present technology, it is possible to increase the degree of freedom in the rewiring line layout or improve imaging characteristics, and further, both an increase in the degree of freedom in the rewiring line layout and improvement of imaging characteristics can be achieved. With the solid-state imaging device of the first embodiment (Example 1 of a solid-state imaging device) according to the present technology, the degree of freedom in rewiring design can be increased, and further, image noise can be reduced by the rewiring line layout in the stack structure.

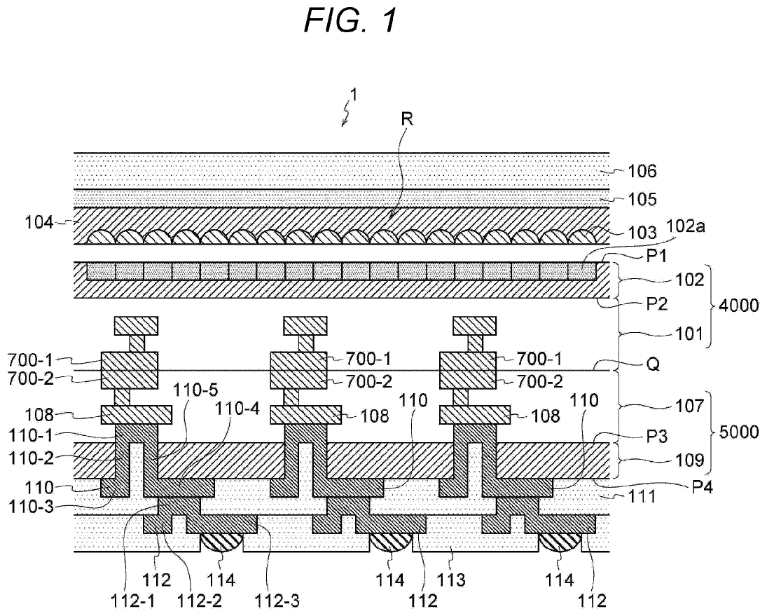

In the description below, a solid-state imaging device of the first embodiment according to the present technology is explained in greater detail, with reference to FIGS. 1 to 10.

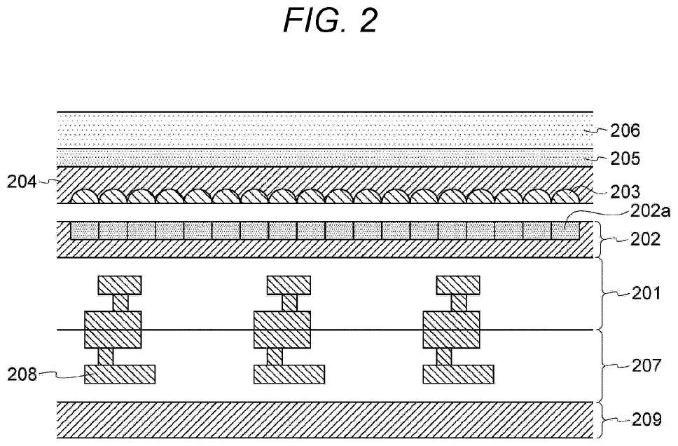

FIG. 1 is a cross-sectional diagram showing an example configuration of a solid-state imaging device of the first embodiment according to the present technology, and more particularly, is a cross-sectional diagram showing the structure of a solid-state imaging device of the first embodiment having a chip-sized package structure including rewiring lines of a stack structure. FIGS. 2 to 10 are cross-sectional views for explaining a method for manufacturing a solid-state imaging device of the first embodiment according to the present technology, and more particularly, are cross-sectional views for explaining a method for manufacturing a solid-state imaging device of the first embodiment having a chip-sized package structure including rewiring lines of a stack structure.

First, explanation is made with reference to FIG. 1. FIG. 1 shows a solid-state imaging device 1. The solid-state imaging device 1 includes, as principal components, a sensor substrate 4000 including a first semiconductor substrate 102 and a first wiring layer 101, a circuit substrate 5000 including a second semiconductor substrate 109 and a second wiring layer 107, a first rewiring line 110, and a second rewiring line 112.

In the solid-state imaging device 1 shown in FIG. 1, light receiving elements (photodiodes in the photoelectric conversion region, for example) 102a for obtaining an imaging signal are formed in a first principal surface P1 of the first semiconductor substrate 102. Microlenses 103 arranged in a light receiving region R are then disposed on the light receiving elements (the photodiodes formed in the photoelectric conversion region, for example) 102a, a planarizing layer 104 for planarizing the portion on the microlenses 103 is formed, and a light transmissive substrate (a transparent protective substrate, for example) 106 is disposed on the planarizing layer 104 via an adhesive layer 105.

The circuit substrate 5000 that receives signals from pixels and performs signal processing, and includes the second semiconductor substrate 109 (also called the circuit substrate silicon unit) and the second wiring layer (also called the circuit substrate wiring unit) 107 formed on a third principal surface P3 of the second semiconductor substrate is connected to the lower portion (the lower side in FIG. 1) of the first wiring layer (also called the image sensor substrate wiring unit) 102 formed on a second principal surface P2 of the first semiconductor substrate 102, and the second wiring layer (the circuit substrate wiring unit) 107 and the first wiring layer (the image sensor substrate wiring unit) 101 are connected to each other via connecting portions 700-1 and 700-2, so that the sensor substrate 4000 and the circuit substrate 5000 are electrically connected to each other.

Further, an internal electrode 108 is formed in the second wiring layer (the circuit substrate wiring unit) 107, and the first rewiring line 110 is formed through the second semiconductor substrate (the circuit substrate silicon unit) 109 and the second wiring layer (the circuit substrate wiring unit) 107. On the first rewiring line 110, the second rewiring line 112 is formed on the side of a fourth principal surface P4 of the second semiconductor substrate via a first interlayer insulating film 111. At a lower portion of the second rewiring line 112, a solder bump 114 is formed in an insulating film opening formed in part of a second interlayer insulating film 113. The solder bump 114 serves as a connecting contact to an external module substrate, enabling formation of a solid-state imaging device including rewiring lines of a two-layer structure formed with the first rewiring line 110 and the second rewiring line 112. The first interlayer insulating film 111 and the second interlayer insulating film 113 may include the same material, or may include different materials.

The first rewiring line 110 includes a first member 110-1, a third member 110-3, and a fourth member 110-4 that are substantially parallel to the third principal surface P3 and the fourth principal surface P4 of the second semiconductor substrate 109, and a second member 110-2 and a fifth member 110-5 that are substantially perpendicular to the third principal surface P3 and the fourth principal surface P4 of the second semiconductor substrate.

The second rewiring line 112 includes a first member 112-1, a second member 112-2, and a third member 112-3 that are substantially parallel to the fourth principal surface P4 of the second semiconductor substrate 109.

The first member 110-1 of the first rewiring line 110 and the internal electrode 108 are then connected, the fourth member 110-4 of the first rewiring line and the first member 112-1 of the second rewiring line are connected, and the second rewiring line 112-3 and the solder bump 114 are connected.

In the solid-state imaging device 1 of the first embodiment according to the present technology, rewiring lines of a two-layer structure formed with the first rewiring line 110 and the second rewiring line 112 is formed in rewiring, and thus, the degree of freedom in layout can be increased. Further, in the solid-state imaging device 1 of the first embodiment according to the present technology, as the degree of freedom in rewiring design becomes higher, a solder bump can be disposed in a desired region in each chip, and the connection strength can be advantageously increased at the time of joining with the module, for example.

Here, it is necessary to use a particular manufacturing method in formation of the rewiring lines of the two-layer structure in the solid-state imaging device 1 described above. In the description below, a method for manufacturing the solid-state imaging device 1 is explained in detail, with reference to FIGS. 2 to 10.

First, as shown in FIG. 2, a structure in which the sensor substrate 4000 that outputs an image signal by photoelectric conversion and the circuit substrate 5000 that receives a signal from the sensor substrate 4000 and performs signal processing are connected to each other is prepared. The sensor substrate 4000 and the circuit substrate 5000 are electrically connected to each other via wiring layers (a first wiring layer 201 and a second wiring layer 207) formed in the respective substrates. A light transmissive substrate (a transparent protective substrate, for example) 206 is formed on a planarizing film 204 on microlenses 203 formed in a portion (the upper side in FIG. 2) above light receiving elements (photodiodes (PDs) formed in the photoelectric conversion region, for example) 202a, via an adhesive layer 205.

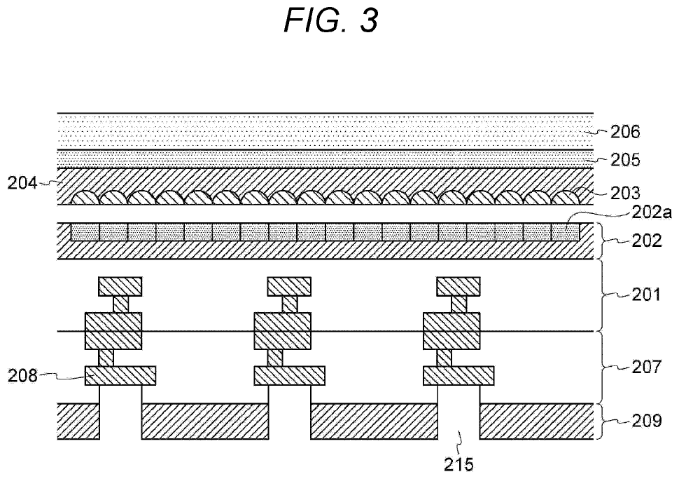

Next, a through hole 215 is formed for an internal electrode 208 formed in the second wiring layer (a circuit substrate wiring unit) 207 (FIG. 3).

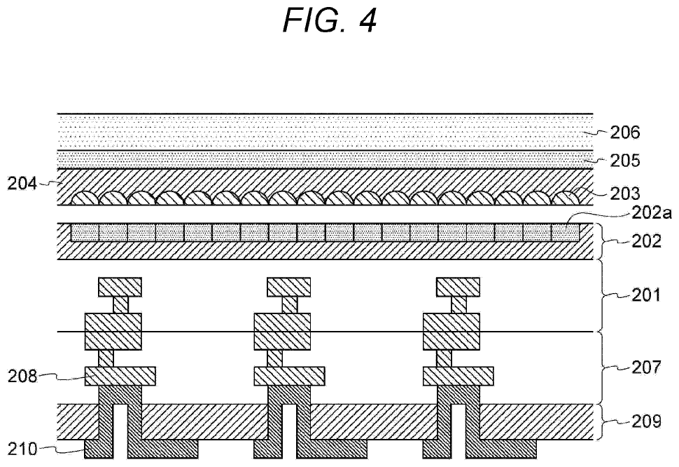

A first rewiring line 210 is then formed in substantially the same layer (FIG. 4). Note that, although not shown in FIG. 4, an insulating film may be formed at the boundary portion between the first rewiring line 210 and a second semiconductor substrate (a circuit substrate silicon unit) 209 so as not to cause electrical short-circuiting. The first rewiring line 210 is formed with a copper film or the like by an electrolytic plating technique using a mask including a resist material having an opening pattern in a desired region, and the film thickness of the first rewiring line 210 is several microns to several tens of microns.

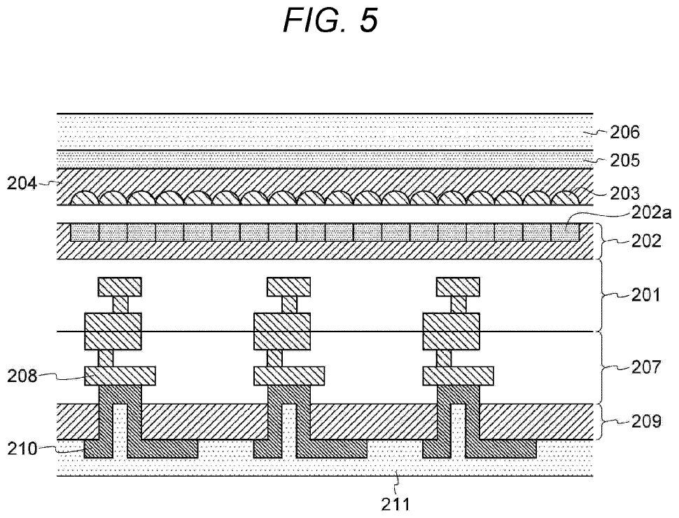

Next, the first rewiring line 210 is covered with a first interlayer insulating film 211 (FIG. 5). A solder resist or the like that is an organic material may be used as the first interlayer insulating film 211. Further, in the next step, a photosensitive solder resist is preferably used as the first interlayer insulating film 211, to form an insulating film opening serving as the portion connecting to a second rewiring line 212.

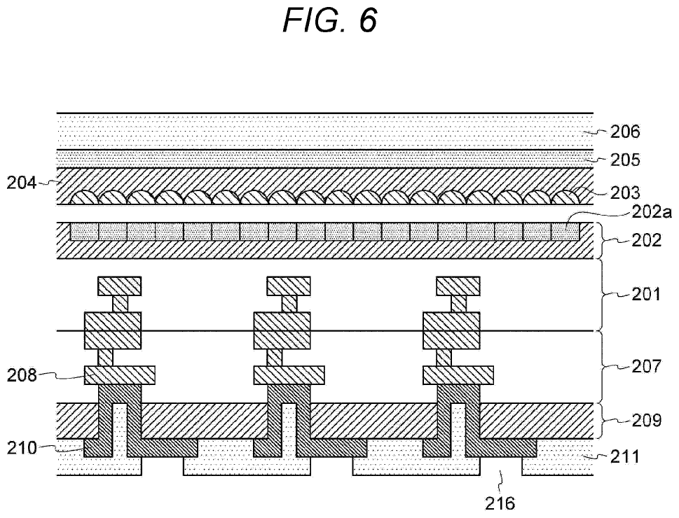

An insulating film opening 216 is then formed in the first interlayer insulating film 211 (FIG. 6). In a case where the first interlayer insulating film 211 is a photosensitive material, an opening can be formed by a lithographic technique at this stage. In a case where a material film that is not a photosensitive material is used, an opening can be formed by dry etching using a mask that is a resist pattern formed by a lithographic technique.

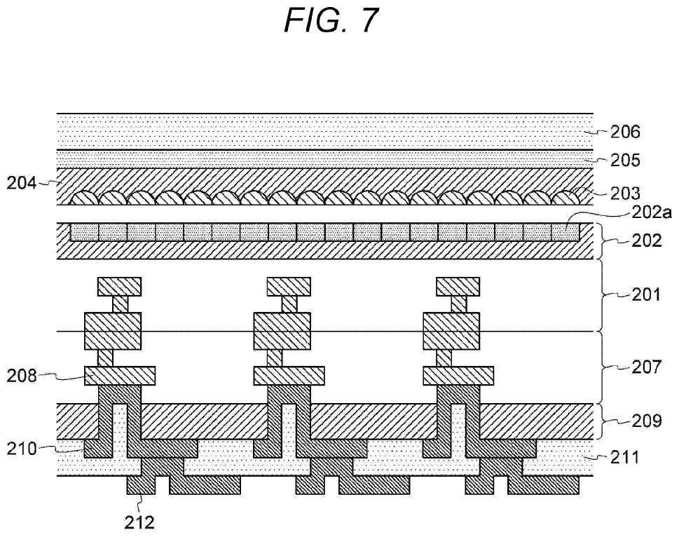

Next, the second rewiring line 212 is formed in substantially the same layer (FIG. 7). The second rewiring line 212 is formed with a copper film or the like by an electrolytic plating technique using a mask including a resist material having an opening pattern in a predetermined region, and the film thickness of the second rewiring line 212 is several microns to several tens of microns.

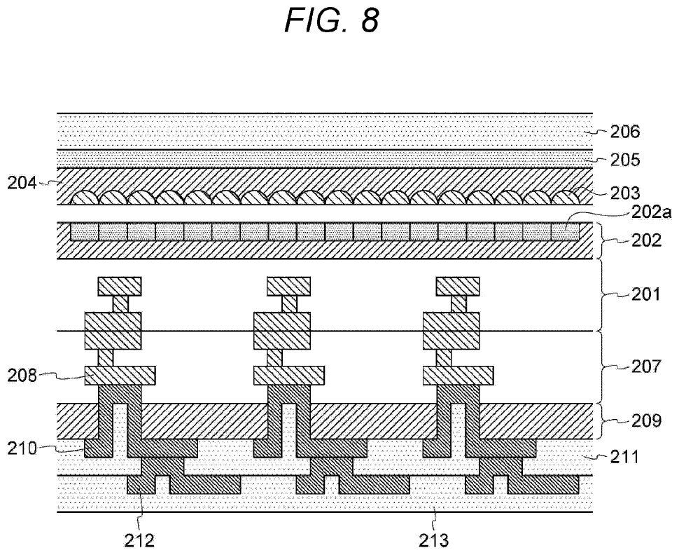

The second rewiring line 212 is then covered with a second interlayer insulating film 213 (FIG. 8). A solder resist or the like that is an organic material may be used as the second interlayer insulating film 213. Further, in the next step, a photosensitive solder resist is preferably used as the second interlayer insulating film 213, to form an insulating film opening for disposing a solder bump or the like to serve as the portion connecting to the module substrate.

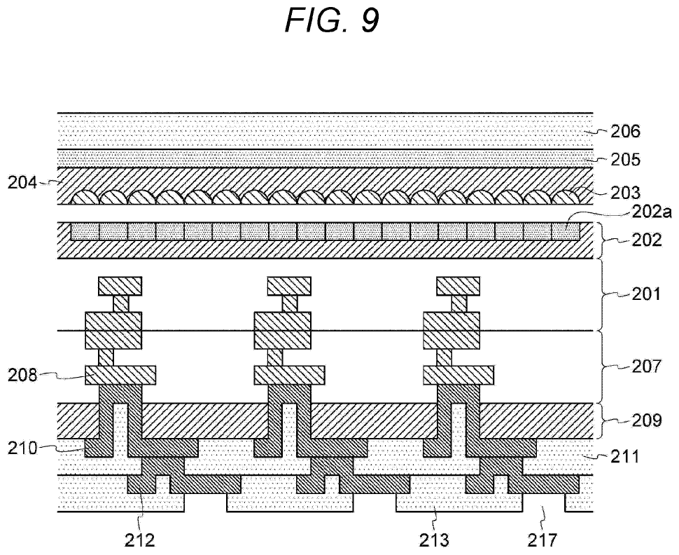

Next, an insulating film opening 217 is formed in the second interlayer insulating film 213 (FIG. 9).

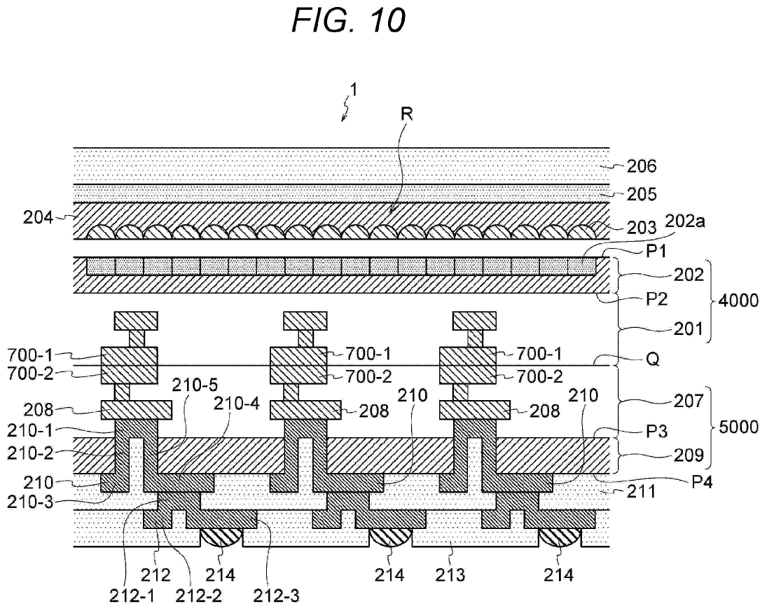

Lastly, a solder bump 214 is formed on the second rewiring line 212 exposed through the insulating film opening formed in the second interlayer insulating film 213, and thus, the solid-state imaging device 1 is completed (FIG. 10).

Here, the solder bump is a joining portion that serves as the contact point between the solid-state imaging device and the module substrate on which the solid-state imaging device is mounted. The solder bump is formed by disposing a ball-like solder material and subjecting the solder material to a heat treatment to shape the solder material into a bump-like form. However, the solder bump may be formed by performing a plating technique on the opening formed in the second interlayer insulating film 213, to form a metallic material such as copper or nickel.

In the formation of the first rewiring line 210 and the second rewiring line 212 of a stack structure of the solid-state imaging device 1 described above, a photosensitive solder resist is used to form a connection opening in the interlayer insulating film 211 covering the first rewiring line 210 and the interlayer insulating film 213 covering the second rewiring line 212. However, this is a manufacturing method that can be used in the formation of the rewiring lines unique to the solid-state imaging device in which the wiring line width of the first rewiring line 210 and/or the second rewiring line 212 is as wide as several tens of microns, and discloses materials and a manufacturing method that differ from those of the micron-order rewiring line formation technology for conventional semiconductor devices.

In addition to the contents described above, the contents that will be explained below in the descriptions of solid-state imaging devices of second to fourth embodiments according to the present technology can be applied, without any change, to the solid-state imaging device of the first embodiment according to the present technology, unless there is some technical contradiction.

### 3. Second Embodiment (Example 2 of a Solid-State Imaging Device)

A solid-state imaging device of a second embodiment (Example 2 of a solid-state imaging device) according to the present technology includes: a sensor substrate that includes a first semiconductor substrate having a first principal surface on the light incident side and a second principal surface on the side opposite from the first principal surface, light receiving elements being formed and arranged two-dimensionally on the first principal surface, and a first wiring layer formed on the second principal surface of the first semiconductor substrate; a circuit substrate that includes a second semiconductor substrate having a third principal surface on the light incident side and a fourth principal surface on the side opposite from the third principal surface, and a second wiring layer formed on the third principal surface of the second semiconductor substrate; a light transmissive substrate disposed above the light receiving elements; a first rewiring line electrically connected to an internal electrode formed in the second wiring layer; and a second rewiring line formed on the side of the fourth principal surface of the second semiconductor substrate. In this solid-state imaging device, the first wiring layer of the sensor substrate and the second wiring layer of the circuit substrate are bonded, to form a stack structure of the sensor substrate and the circuit substrate. Also, in the solid-state imaging device of the second embodiment (Example 2 of a solid-state imaging device) according to the present technology, the first rewiring line is formed with a plurality of first rewiring lines, the second rewiring line is formed with a plurality of second rewiring lines, the wiring layers include a plurality of signal lines formed in a predetermined direction, and the plurality of first rewiring lines and the plurality of second wiring layers are disposed so as to generate a plurality of magnetic fields having different magnetic directions from one another in a region between two adjacent signal lines of the plurality of signal lines.

Further, in the solid-state imaging device of the second embodiment (Example 2 of a solid-state imaging device) according to the present technology, at least one pair of one first rewiring line of the plurality of first rewiring lines and one second rewiring line of the plurality of second rewiring lines is formed in a vertical direction, and the direction of a first current flowing in the one first rewiring line and the direction of a second current flowing in the one second rewiring line are the opposite from each other.

In the solid-state imaging device of the second embodiment (Example 2 of a solid-state imaging device) according to the present technology, the second rewiring line may be disposed below the first rewiring line. Also, in the solid-state imaging device of the second embodiment (Example 2 of a solid-state imaging device) according to the present technology, the first rewiring line may be formed with a plurality of first rewiring lines, the second rewiring line may be formed with a plurality of second rewiring lines, and at least one first rewiring line of the plurality of first rewiring lines and at least one second rewiring line of the plurality of second rewiring lines may be connected.

With the solid-state imaging device of the second embodiment (Example 2 of a solid-state imaging device) according to the present technology, it is possible to increase the degree of freedom in the rewiring line layout or improve imaging characteristics, and further, both an increase in the degree of freedom in the rewiring line layout and improvement of imaging characteristics can be achieved. With the solid-state imaging device of the second embodiment (Example 2 of a solid-state imaging device) according to the present technology, the degree of freedom in rewiring design can be increased, and further, image noise can be reduced by the rewiring line layout in the stack structure.

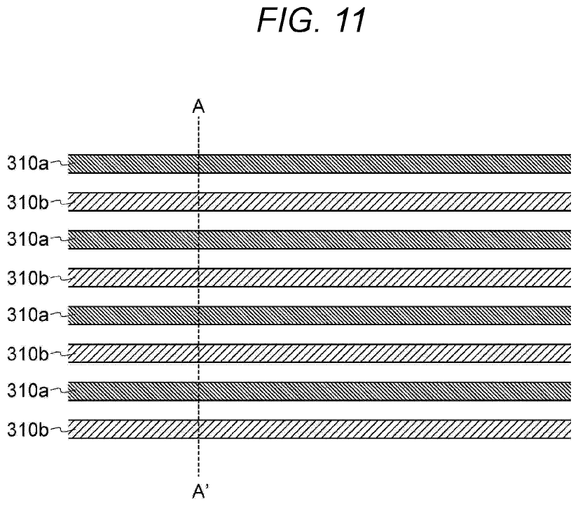

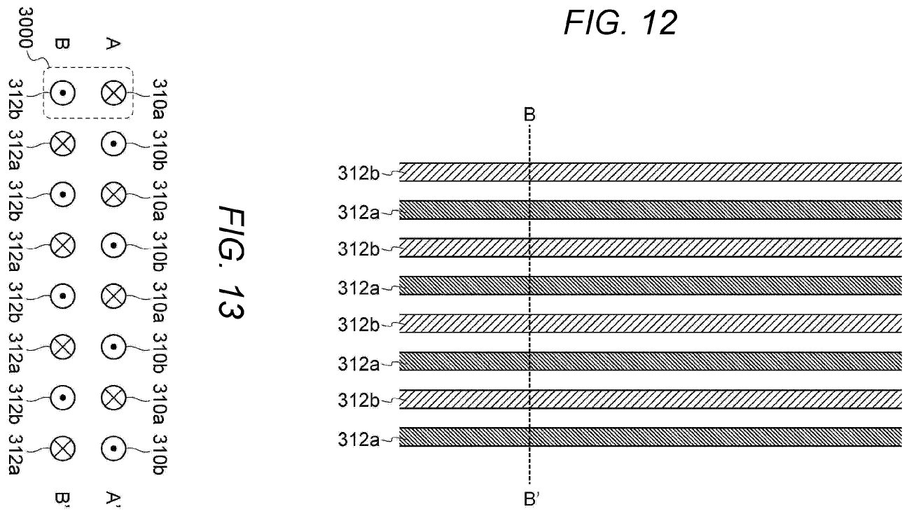

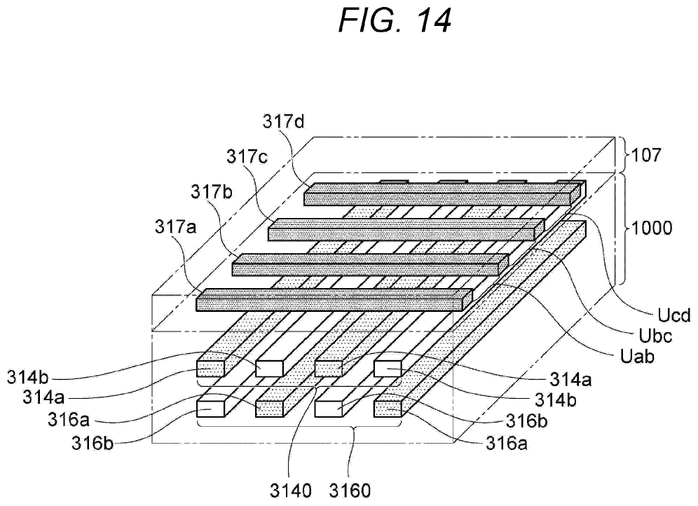

In the description below, a solid-state imaging device of the second embodiment according to the present technology is explained in greater detail, with reference to FIGS. 11 to 14. FIG. 11 is a plan view of an example configuration of a plurality of first rewiring lines included in the solid-state imaging device of the second embodiment according to the present technology. FIG. 12 is a plan view of an example configuration of a plurality of second rewiring lines included in the solid-state imaging device of the second embodiment according to the present technology. FIG. 13 is a cross-sectional diagram showing an example configuration of a plurality of first rewiring lines and an example configuration of a plurality of second rewiring lines included in the solid-state imaging device of the second embodiment according to the present technology. FIG. 14 is a perspective view of an example configuration of a plurality of first rewiring lines, a plurality of second rewiring lines, and a plurality of signal lines included in the solid-state imaging device of the second embodiment according to the present technology.

In the solid-state imaging device of the second embodiment having rewiring lines of a stack structure, a layout unique to a solid-state imaging device can be used in the rewiring line arrangement pattern. First, explanation is made with reference to FIG. 11.

FIG. 11 schematically shows a planar layout of first rewiring lines, and power-supply wiring lines 310a and GND wiring lines 310b are alternately arranged in FIG. 11.

FIG. 12 schematically shows a planar layout of second rewiring lines, and power-supply wiring lines 312a and GND wiring lines 312b are alternately arranged in FIG. 12.

FIG. 13 shows a schematic cross-sectional view corresponding to an A-A′ line shown in FIG. 11 and a B-B′ line shown in FIG. 12. FIG. 13 schematically shows a situation where electric current flows from the back to the front of the drawing In the wiring lines having ⋅ (dots) in the circles of cross-sections of the power-supply wiring lines 310a and 312a, and the GND wiring lines 310b and 312b, and electric current flows from the front to the back of the drawing in the wiring lines having × in the circles. As shown in FIG. 13, the directions of flowing electric current are the opposite from each other in one pair 3000 formed with a first rewiring line 310a and a second rewiring line 312b adjacent to each other in the vertical direction (the vertical direction in FIG. 13), for example. Note that FIG. 13 discloses that the current directions differ between the first rewiring lines and the second rewiring lines that are adjacent in the vertical direction and form all the pairs, but some of the first rewiring lines may have a different current direction from that of the other first rewiring lines.

In the layout of the first rewiring lines and the second rewiring lines shown in FIGS. 11 to 13, the magnetic field generated by the current application in each rewiring line, and the magnetic field generated in each adjacent rewiring line (for example, a first rewiring line and a second rewiring line adjacent to each other in the vertical direction, first wiring lines adjacent to each other in the horizontal direction, or second wiring lines adjacent to each other in the horizontal direction) can be canceled each other out. The electromotive force generated in the signal wiring lines for transmitting signal charges extracted from pixels due to the magnetic fields generated in the rewiring lines can be reduced, and thus, image noise resulting from the electromotive force can be reduced.

FIG. 14 shows the configuration of a plurality of first rewiring lines, a plurality of second rewiring lines, and a plurality of signal lines as a specific example. As shown in FIG. 14, a second wiring layer (a circuit substrate wiring unit) 207 is formed, and a rewiring line formation portion 1000 is formed in the lower portion (the lower side in FIG. 14) of the second wiring layer (the circuit substrate wiring unit) 207. A plurality of signal lines 317a to 317d is formed in the second wiring layer (the circuit substrate wiring unit) 207, a plurality of first rewiring line groups 3140 is formed in the rewiring line formation portion 1000, and a plurality of second rewiring line groups 3160 is formed below (the lower side in FIG. 14) the plurality of first rewiring line groups 3140.

As the plurality of first rewiring line groups 3140, power-supply wiring lines 314a and GND wiring lines 314b are alternately arranged from the left side in FIG. 14. As the plurality of second rewiring line groups 3160, GND wiring lines 316b and power-supply wiring lines 316a are alternately arranged from the left side in FIG. 14. That is, when the plurality of first rewiring line groups 3140 and the plurality of second rewiring line groups 3160 are viewed together (viewed vertically (in the vertical direction) in FIG. 14), one pair formed with one power-supply wiring line and one GND wiring line has a two-layer structure, and a plurality of such pairs is provided. As the direction of the current in the power-supply wiring line and the direction of the current in the GND wiring line in a pair are the opposite from each other, the magnetic fields generated by current application can be canceled each other out in the region Uab between the adjacent signal lines 317a and 317b, in the region Ubc between the adjacent signal lines 317b and 317c, and in the region Ucd between the adjacent signal lines 317c and 317d. Thus, image noise can be reduced.

In addition to the contents described above, the contents described in the description of the solid-state imaging device of the first embodiment according to the present technology and the contents that will be explained below in the descriptions of solid-state imaging devices of third and fourth embodiments according to the present technology can be applied, without any change, to the solid-state imaging device of the second embodiment according to the present technology, unless there is some technical contradiction.

### 4. Third Embodiment (Example 3 of a Solid-State Imaging Device)

A solid-state imaging device of a third embodiment (Example 3 of a solid-state imaging device) according to the present technology includes: a sensor substrate that includes a first semiconductor substrate having a first principal surface on the light incident side and a second principal surface on the side opposite from the first principal surface, light receiving elements being formed and arranged two-dimensionally on the first principal surface, and a first wiring layer formed on the second principal surface of the first semiconductor substrate; a circuit substrate that includes a second semiconductor substrate having a third principal surface on the light incident side and a fourth principal surface on the side opposite from the third principal surface, and a second wiring layer formed on the third principal surface of the second semiconductor substrate; a light transmissive substrate disposed above the light receiving elements; a first rewiring line electrically connected to an internal electrode formed in the second wiring layer; and a second rewiring line formed on the side of the fourth principal surface of the second semiconductor substrate. In this solid-state imaging device, the first wiring layer of the sensor substrate and the second wiring layer of the circuit substrate are bonded, to form a stack structure of the sensor substrate and the circuit substrate. Further, in the solid-state imaging device of the third embodiment (Example 3 of a solid-state imaging device) according to the present technology, the first rewiring line is formed with a plurality of first rewiring lines, the second rewiring line is formed with a plurality of second rewiring lines, the wiring layers include a plurality of signal lines formed in a predetermined direction, and at least one first rewiring line of the plurality of first rewiring lines and/or at least one second rewiring line of the plurality of second rewiring lines covers at least part of at least one signal line of the plurality of signal lines, when viewed from the side opposite from the light incident side.

In the solid-state imaging device of the third embodiment (Example 3 of a solid-state imaging device) according to the present technology, the second rewiring line may be disposed below the first rewiring line. Also, in the solid-state imaging device of the third embodiment (Example 3 of a solid-state imaging device) according to the present technology, the first rewiring line may be formed with a plurality of first rewiring lines, the second rewiring line may be formed with a plurality of second rewiring lines, and at least one first rewiring line of the plurality of first rewiring lines and at least one second rewiring line of the plurality of second rewiring lines may be connected.

With the solid-state imaging device of the third embodiment (Example 3 of a solid-state imaging device) according to the present technology, it is possible to increase the degree of freedom in the rewiring line layout or improve imaging characteristics, and further, both an increase in the degree of freedom in the rewiring line layout and improvement of imaging characteristics can be achieved. With the solid-state imaging device of the third embodiment (Example 3 of a solid-state imaging device) according to the present technology, the degree of freedom in rewiring design can be increased, and further, image noise can be reduced by the rewiring line layout in the stack structure.

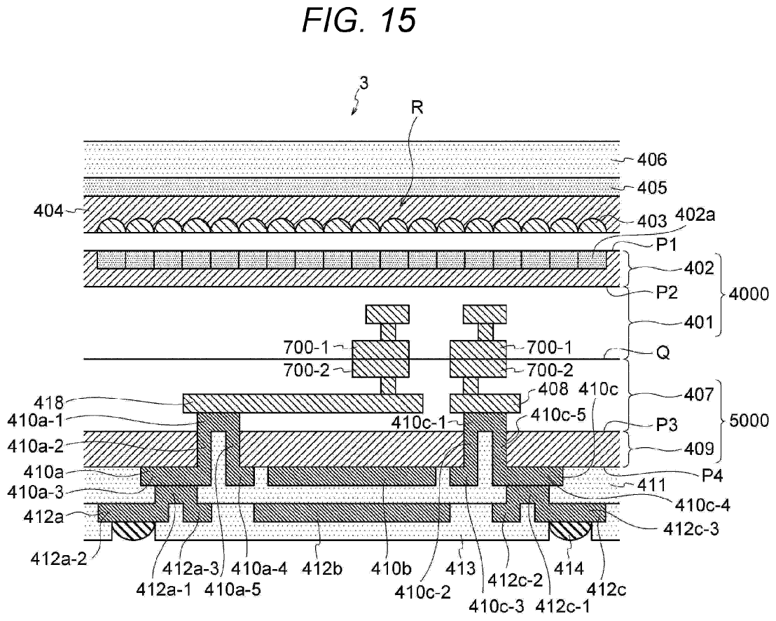

In the description below, a solid-state imaging device of the third embodiment according to the present technology is explained in greater detail, with reference to FIG. 15. FIG. 15 is a cross-sectional diagram showing an example configuration of a solid-state imaging device of the second embodiment according to the present technology, and more particularly, is a cross-sectional diagram showing the structure of a solid-state imaging device of the second embodiment having a chip-sized package structure including rewiring lines of a stack structure.

This is now explained with reference to FIG. 15. FIG. 15 shows a solid-state imaging device 3.

FIG. 15 shows a signal line 418 that is formed in a second wiring layer (a circuit substrate wiring unit) 407, and is designed for transmitting signal charge from a sensor substrate 4000. As shown in FIG. 15, the first rewiring line 410b and a second rewiring line 412b are disposed in a region immediately below the signal line 418, and are arranged so as to spatially overlap at least part of the region of the signal line 418. That is, the first rewiring line 410b and the second rewiring line 412b cover at least part of the signal line 418, when viewed from the opposite side (the front surface side, which is the lower side in FIG. 15) from the light incident side (the back surface side, which is the upper side in FIG. 15). Here, the first rewiring line 410b and the second rewiring line 412b disposed immediately below the signal line 418 may be fixed to the ground potential or the like at a location not shown in FIG. 15.

A first rewiring line 410a includes a first member 410a-1, a third member 410a-3, and a fourth member 410a-4 that are substantially parallel to a third principal surface P3 and a fourth principal surface P4 of a second semiconductor substrate 409, and a second member 410a-2 and a fifth member 410a-5 that are substantially perpendicular to the third principal surface P3 and the fourth principal surface P4 of the second semiconductor substrate 409.

A second rewiring line 412a includes a first member 412a-1, a second member 412a-2, and a third member 412a-3 that are substantially parallel to the fourth principal surface P4 of the second semiconductor substrate 409.

The first member 410a-1 of the first rewiring line 410a and the signal line 418 are then connected, the fourth member 410a-4 of the first rewiring line and the first member 412a-1 of the second rewiring line are connected, and the second rewiring line 412a-3 and a solder bump 414 are connected.

Further, a first rewiring line 410c includes a first member 410c-1, a third member 410c-3, and a fourth member 410c-4 that are substantially parallel to the third principal surface P3 and the fourth principal surface P4 of the second semiconductor substrate 409, and a second member 410c-2 and a fifth member 410c-5 that are substantially perpendicular to the third principal surface P3 and the fourth principal surface P4 of the second semiconductor substrate 409.

A second rewiring line 412c includes a first member 412c-1, a second member 412c-2, and a third member 412c-3 that are substantially parallel to the fourth principal surface P4 of the second semiconductor substrate 409.

The first member 410c-1 of the first rewiring line 410c and an internal electrode 408 are then connected, the fourth member 410c-4 of the first rewiring line and the first member 412c-1 of the second rewiring line are connected, and the second rewiring line 412c-3 and the solder bump 414 are connected.

The layout of the first rewiring line 410b and the second rewiring line 412b shown in FIG. 15 shields the signal line 418 from the influence of a magnetic field from the module substrate connected to the solid-state imaging device 3, for example, and thus, noise in pixel signals can be reduced. Further, as the first rewiring line 410b and the second rewiring line 412b are disposed in the region immediately below the power supply for pixels, the GND wiring line, and the like, magnetic fields from the solid-state imaging device 3 can be prevented from affecting the circuits mounted on the module substrate.

In addition to the contents described above, the contents described in the descriptions of the solid-state imaging devices of the first and second embodiments according to the present technology and the contents that will be explained below in the description of a solid-state imaging device of the fourth embodiment according to the present technology can be applied, without any change, to the solid-state imaging device of the third embodiment according to the present technology, unless there is some technical contradiction.

### 5. Fourth Embodiment (Example 4 of a Solid-State Imaging Device)

A solid-state imaging device of a fourth embodiment (Example 4 of a solid-state imaging device) according to the present technology includes: a sensor substrate that includes a first semiconductor substrate having a first principal surface on the light incident side and a second principal surface on the side opposite from the first principal surface, light receiving elements being formed and arranged two-dimensionally on the first principal surface, and a first wiring layer formed on the second principal surface of the first semiconductor substrate; a circuit substrate that includes a second semiconductor substrate having a third principal surface on the light incident side and a fourth principal surface on the side opposite from the third principal surface, and a second wiring layer formed on the third principal surface of the second semiconductor substrate; a light transmissive substrate disposed above the light receiving elements; a first rewiring line electrically connected to an internal electrode formed in the second wiring layer; and a second rewiring line formed on the side of the fourth principal surface of the second semiconductor substrate. In this solid-state imaging device, the first wiring layer of the sensor substrate and the second wiring layer of the circuit substrate are bonded, to form a stack structure of the sensor substrate and the circuit substrate. Also, in the solid-state imaging device of the fourth embodiment (Example 4 of a solid-state imaging device) according to the present technology, the first rewiring line is formed with a plurality of first rewiring lines, a groove portion is formed on the side of the fourth principal surface of the second semiconductor substrate, and at least part of at least one first rewiring line of the plurality of first rewiring lines is formed in the groove portion.

Further, in the solid-state imaging device of the fourth embodiment (Example 4 of a solid-state imaging device) according to the present technology, the depth of the groove portion is preferably not smaller than the thickness of at least one first rewiring line of the plurality of first rewiring lines.

In the solid-state imaging device of the fourth embodiment (Example 4 of a solid-state imaging device) according to the present technology, the second rewiring line may be disposed below the first rewiring line. Also, in the solid-state imaging device of the fourth embodiment (Example 4 of a solid-state imaging device) according to the present technology, the first rewiring line may be formed with a plurality of first rewiring lines, the second rewiring line may be formed with a plurality of second rewiring lines, and at least one first rewiring line of the plurality of first rewiring lines and at least one second rewiring line of the plurality of second rewiring lines may be connected.

With the solid-state imaging device of the fourth embodiment (Example 4 of a solid-state imaging device) according to the present technology, it is possible to increase the degree of freedom in the rewiring line layout or improve imaging characteristics, and further, both an increase in the degree of freedom in the rewiring line layout and improvement of imaging characteristics can be achieved. With the solid-state imaging device of the fourth embodiment (Example 4 of a solid-state imaging device) according to the present technology, the degree of freedom in rewiring design can be increased, and further, image noise can be reduced by the rewiring line layout in the stack structure.

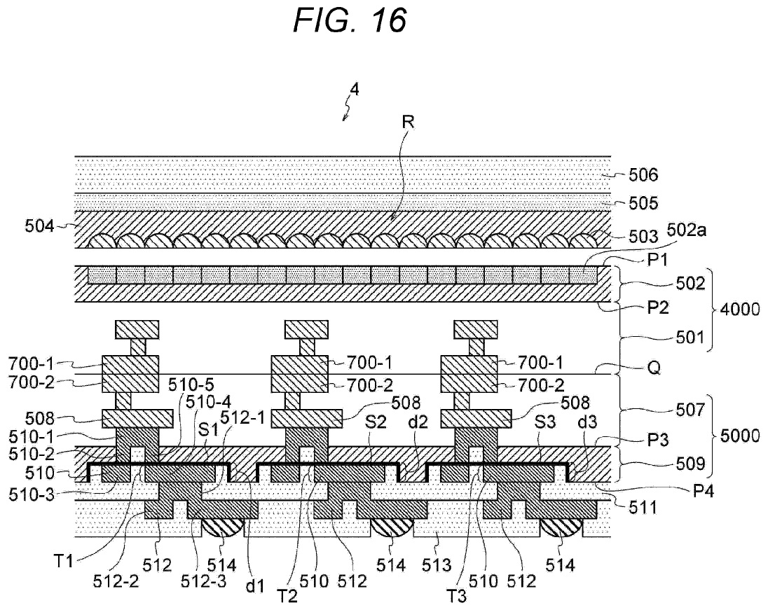

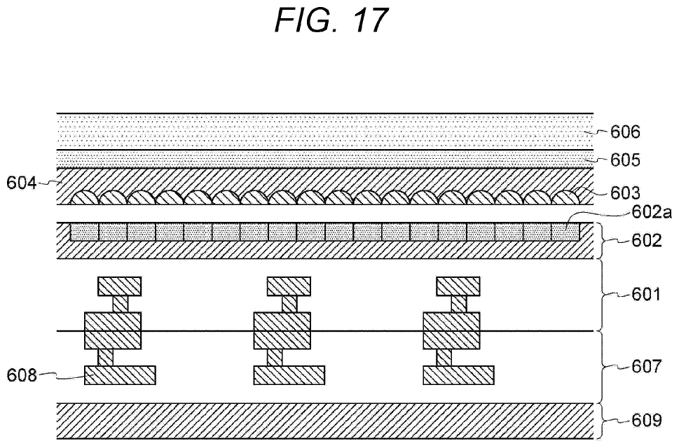

In the description below, a solid-state imaging device of the fourth embodiment according to the present technology is explained in greater detail, with reference to FIGS. 16 to 26. FIG. 16 is a cross-sectional diagram showing an example configuration of a solid-state imaging device of the fourth embodiment according to the present technology, and more particularly, is a cross-sectional diagram showing the structure of a solid-state imaging device of the fourth embodiment having a chip-sized package structure including rewiring lines of a stack structure. FIGS. 17 to 26 are cross-sectional views for explaining a method for manufacturing a solid-state imaging device of the fourth embodiment according to the present technology, and more particularly, are cross-sectional views for explaining a method for manufacturing a solid-state imaging device of the fourth embodiment having a chip-sized package structure including rewiring lines of a stack structure.

First, explanation is made with reference to FIG. 16. FIG. 16 shows a solid-state imaging device 4. In the solid-state imaging device 4, when the rewiring lines are turned into a stack structure, the thickness of the solid-state imaging device increases by the amount equivalent to the thickness of the rewiring lines and the thickness of the interlayer insulating film between the rewiring lines. As a result, the thickness of an entire camera module using this solid-state imaging device increases. To counter this, it is possible to provide a structure (a lowered structure) in which the thickness of the solid-state imaging device using a stacked rewiring line structure can be reduced.

A difference between the structure of the solid-state imaging device 4 shown in FIG. 16 and the structure of the solid-state imaging device 1 shown in FIG. 1 is that silicon recess portions S1 to S3 are formed in a second wiring layer (a circuit substrate silicon unit) 509 that serves as the formation region for three first rewiring lines 510. In FIG. 16, the silicon recess portions S-1 to S-3 have depths d1 to d3 that are equal to or greater than the thicknesses T1 to T3 of the first rewiring lines 510. Note that the depths d1 to d3 of the silicon recess portions S1 to S3 may be smaller than the thicknesses T1 to T3 of the first rewiring lines 510.

With the structure of the solid-state imaging device 4 according to this embodiment, it is possible to reduce the increase in the thickness of the solid-state imaging device even in a case where stacked rewiring lines are adopted, and thus, the thickness of an entire camera module using this solid-state imaging device can also be reduced.

A first rewiring line 510 includes a first member 510-1, a third member 510-3, and a fourth member 510-4 that are substantially parallel to a third principal surface P3 and a fourth principal surface P4 of the second semiconductor substrate 509, and a second member 510-2 and a fifth member 510-5 that are substantially perpendicular to the third principal surface P3 and the fourth principal surface P4 of the second semiconductor substrate 509.

A second rewiring line 512 includes a first member 512-1, a second member 512-2, and a third member 512-3 that are substantially parallel to the fourth principal surface P4 of the second semiconductor substrate 509.

The first member 510-1 of the first rewiring line 510 and an internal electrode 508 are then connected, the fourth member 510-4 of the first rewiring line and the first member 512-1 of the second rewiring line are connected, and the second rewiring line 512-3 and a solder bump 514 are connected.

Specifically, the thickness T1 of the first rewiring line 510 is the thickness (the length in the vertical direction in FIG. 16) of the third member 510-3 or/and the fourth member 510-4 of the first rewiring line 510 (the same applies to T2 and T3).

Next, a method for manufacturing the solid-state imaging device 4 is described with reference to FIGS. 17 to 26.

First, as shown in FIG. 17, a structure in which the sensor substrate 4000 that outputs an image signal by photoelectric conversion and the circuit substrate 5000 that receives a signal from the sensor substrate 4000 and performs signal processing are connected to each other is prepared. The sensor substrate 4000 and the circuit substrate 5000 are electrically connected via wiring portions (a first wiring layer 601 and a second wiring layer 607) formed in the respective substrates, and a light transmissive substrate (a transparent protective substrate, for example) 606 is formed, via an adhesive layer 605, on a planarizing film 604 on microlenses 603 formed above a photoelectric conversion region 602.

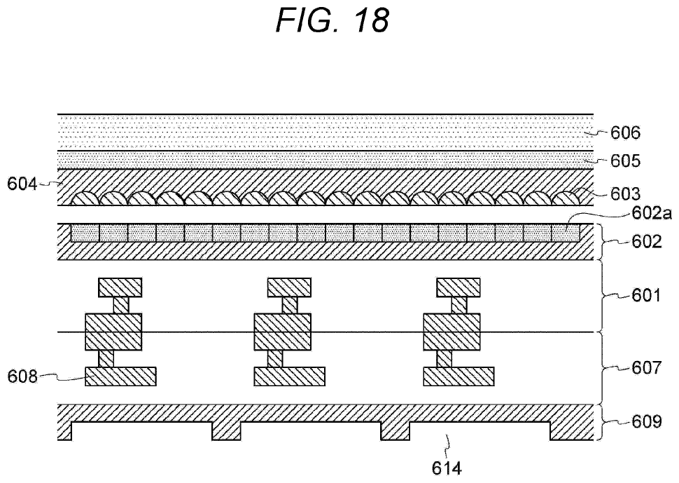

Next, a silicon recess portion 614 is formed in a second semiconductor substrate (a circuit substrate silicon unit) 609 (FIG. 18).

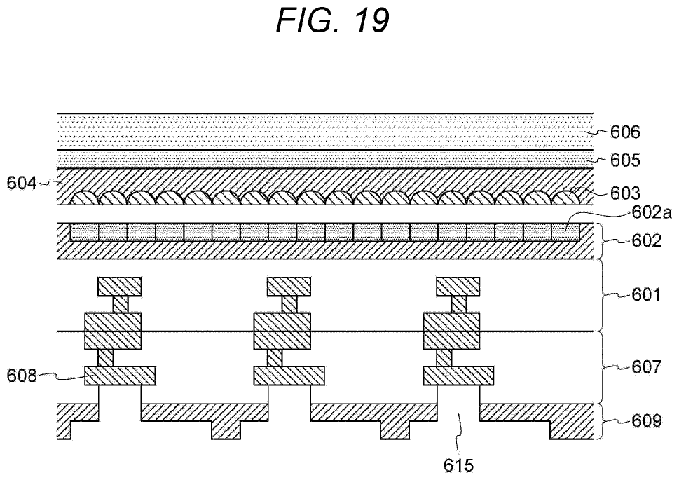

Next, a through hole 615 is formed for an internal electrode 608 formed in the second wiring layer (a circuit substrate wiring unit) 607 (FIG. 19).

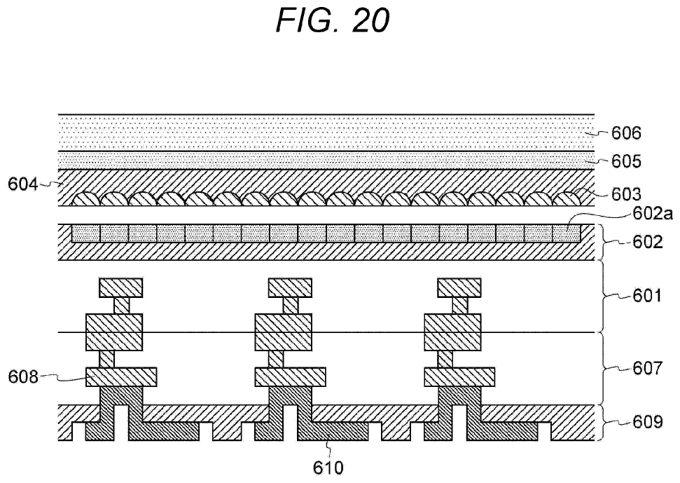

A first rewiring line 610 is then formed in substantially the same layer (FIG. 20). Note that, although not shown in FIG. 20, an insulating film may be formed at the boundary portion between the first rewiring line 610 and the second semiconductor substrate (the circuit substrate silicon unit) 609 so as not to cause electrical short-circuiting. The first rewiring line 610 is formed with a copper film or the like by an electrolytic plating technique using a mask including a resist material having an opening pattern in a predetermined region, and the film thickness of the first rewiring line is several microns to several tens of microns.

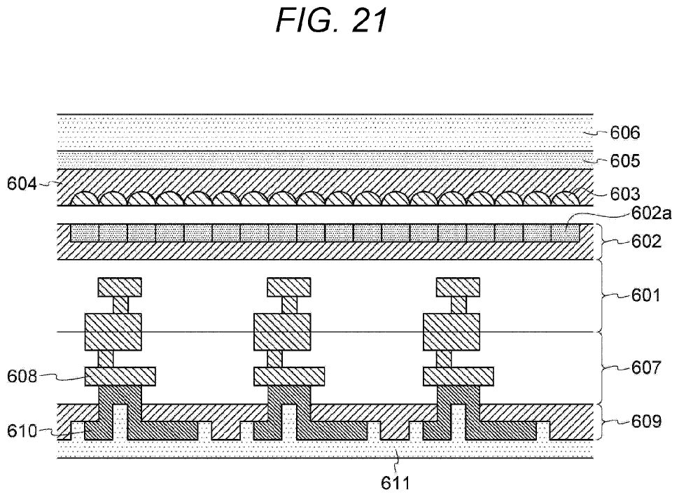

Next, the first rewiring line 610 is covered with a first interlayer insulating film 611 (FIG. 21). A solder resist or the like that is an organic material may be used as the first interlayer insulating film 611. Further, in the next step, a photosensitive solder resist is preferably used as the first interlayer insulating film 611, to form an insulating film opening serving as the portion connecting to a second rewiring line 612.

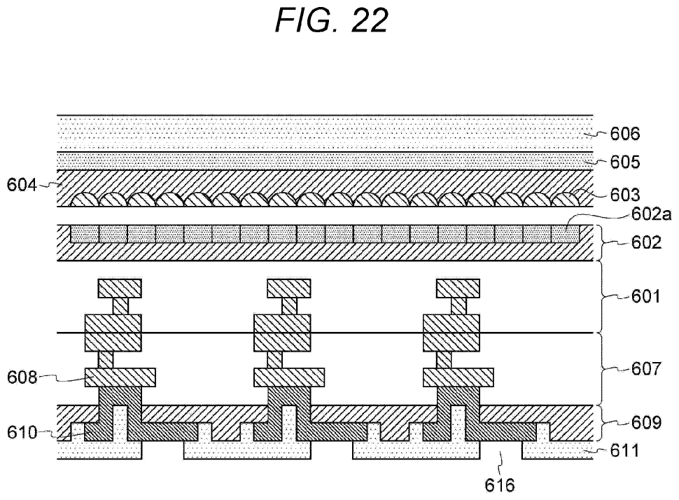

An insulating film opening 616 is then formed in the first interlayer insulating film 611 (FIG. 22). In a case where the first interlayer insulating film 611 is a photosensitive material, an opening can be formed by a lithographic technique at this stage. In a case where a material film that is not a photosensitive material is used, an opening can be formed by dry etching using a mask that is a resist pattern formed by a lithographic technique.

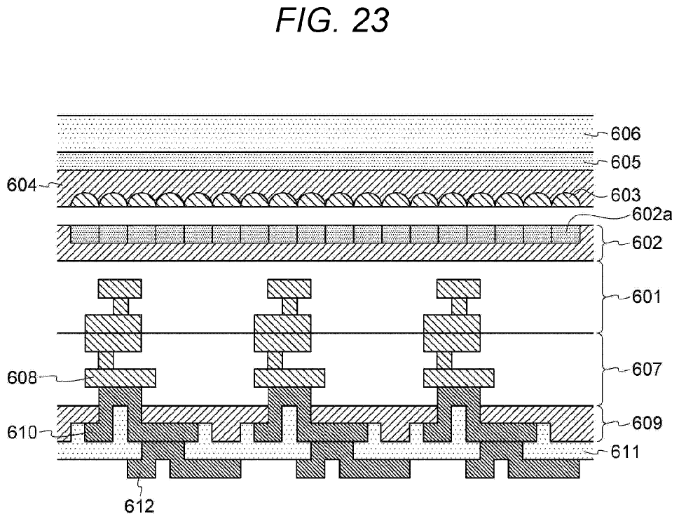

Next, the second rewiring line 612 is formed in substantially the same layer (FIG. 23). The second rewiring line 612 is formed with a copper film or the like by an electrolytic plating technique using a mask including a resist material having an opening pattern in a predetermined region, and the film thickness of the second rewiring line is several microns to several tens of microns.

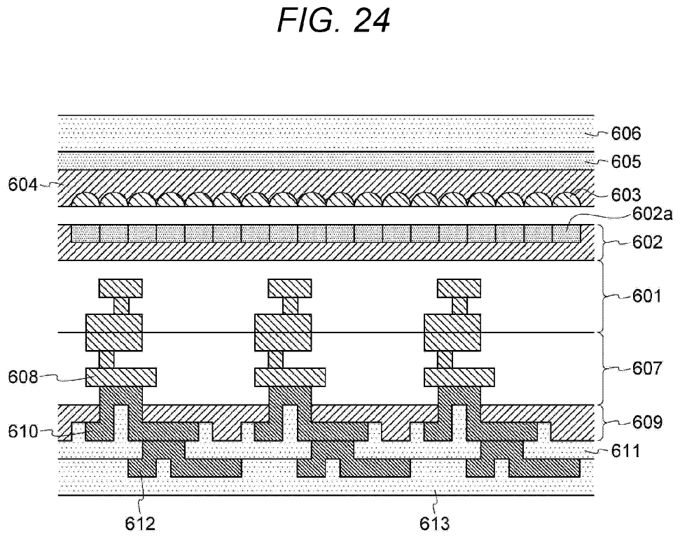

The second rewiring line 612 is then covered with a second interlayer insulating film 613 (FIG. 24). A solder resist or the like that is an organic material may be used as the second interlayer insulating film 613. Further, in the next step, a photosensitive solder resist is preferably used as the second interlayer insulating film 613, to form an insulating film opening for disposing a solder bump or the like to serve as the portion connecting to the module substrate.

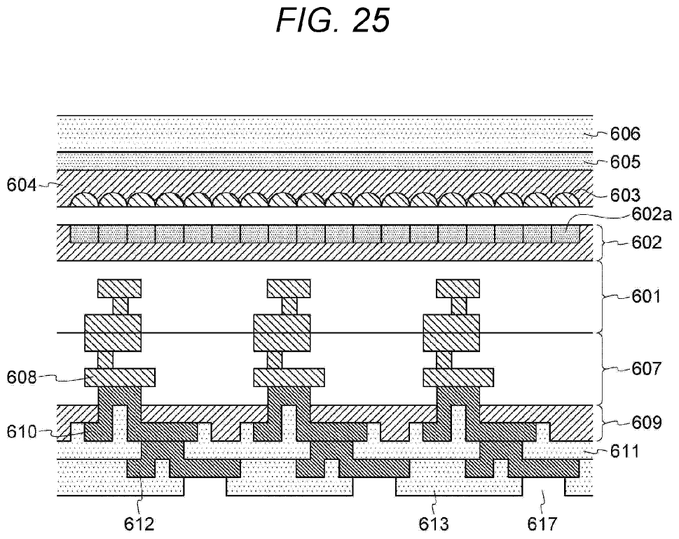

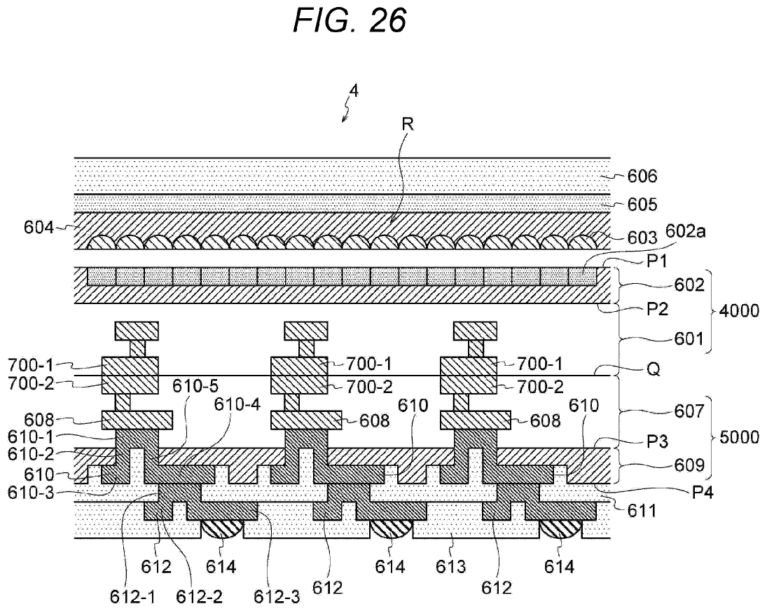

Next, an insulating film opening 617 is formed in the second interlayer insulating film 613 (FIG. 25). Lastly, a solder bump 614 is formed on the second rewiring line 612 exposed through the insulating film opening formed in the second interlayer insulating film 613, and thus, the solid solid-state imaging device 4 is completed (FIG. 26). Here, instead of the solder bump, a metallic material such as copper or nickel may be formed by a plating technique, as in the solid-state imaging device 1 of the first first embodiment according to the present technology.

In the formation of the rewiring lines of the stack structure in the solid-state imaging device 4 of the fourth embodiment according to the present technology, a silicon recess portion is formed in the second semiconductor substrate (the circuit substrate silicon unit) 609 serving as the formation region of the first rewiring line 610, so that the thickness of the solid-state imaging device can be reduced by the amount equivalent to the thickness of the first rewiring line 610, and the thickness of the entire camera module can be reduced. Thus, the apparatus using the camera module can be made smaller (particularly, the height can be reduced).

Further, it is possible to shorten the processing time of the dry etching process when the through hole serving as the connecting portion between the first rewiring line 610 and the internal electrode 608 is formed. Thus, the plasma damage to be caused by the dry etching process can be reduced, and fluctuations in the device characteristics of solid-state imaging devices and the transistors and the like formed in the circuit substrates can also be reduced.

In addition to the contents described above, the contents explained in the descriptions of the solid-state imaging devices of the first to third embodiments according to the present technology can be applied, without any change, to the solid-state imaging device of the fourth embodiment according to the present technology, unless there is some technical contradiction.

### 6. Fifth Embodiment (Example 5 of a Solid-State Imaging Device)

A solid-state imaging device of a fifth embodiment (Example 5 of a solid-state imaging device) according to the present technology is a solid-state imaging device that includes: a semiconductor substrate that has a first principal surface on the light incident side and a second principal surface on the side opposite from the first principal surface, light receiving elements being arranged two-dimensionally on the first principal surface; a light transmissive substrate disposed above the light receiving elements; a wiring layer formed on the second principal surface of the semiconductor substrate; a first rewiring line electrically connected to an internal electrode formed in the wiring layer; and a second rewiring line formed on the side of the second principal surface of the semiconductor substrate.

In the solid-state imaging device of the fifth embodiment (Example 5 of a solid-state imaging device) according to the present technology, the second rewiring line may be disposed below the first rewiring line. Also, in the solid-state imaging device of the fifth embodiment (Example 5 of a solid-state imaging device) according to the present technology, the first rewiring line may be formed with a plurality of first rewiring lines, the second rewiring line may be formed with a plurality of second rewiring lines, and at least one first rewiring line of the plurality of first rewiring lines and at least one second rewiring line of the plurality of second rewiring lines may be connected.

In the solid-state imaging device of the fifth embodiment (Example 2 of a solid-state imaging device) according to the present technology, the first rewiring line may be formed with a plurality of first rewiring lines, the second rewiring line may be formed with a plurality of second rewiring lines, the wiring layer may include a plurality of signal lines formed in a predetermined direction, and the plurality of first rewiring lines and the plurality of second rewiring lines may be disposed so as to generate a plurality of magnetic fields having different magnetic directions from one another in a region between two adjacent signal lines of the plurality of signal lines.

Further, in the solid-state imaging device of the fifth embodiment (Example 5 of a solid-state imaging device) according to the present technology, at least one pair of one first rewiring line of the plurality of first rewiring lines and one second rewiring line of the plurality of second rewiring lines may be formed in a vertical direction, and the direction of a first current flowing in the one first rewiring line and the direction of a second current flowing in the one second rewiring line may be the opposite from each other.

Also, in the solid-state imaging device of the fifth embodiment (Example 5 of a solid-state imaging device) according to the present technology, the first rewiring line may be formed with a plurality of first rewiring lines, the second rewiring line may be formed with a plurality of second rewiring lines, the wiring layer may include a plurality of signal lines formed in a predetermined direction, and at least one first rewiring line of the plurality of first rewiring lines and/or at least one second rewiring line of the plurality of second rewiring lines may cover at least part of at least one signal line of the plurality of signal lines, when viewed from the side opposite from the light incident side.

Further, in the solid-state imaging device of the fifth embodiment (Example 5 of a solid-state imaging device) according to the present technology, the first rewiring line may be formed with a plurality of first rewiring lines, a groove portion may be formed on the side of the second principal surface of the semiconductor substrate, and at least part of at least one first rewiring line of the plurality of first rewiring lines may be formed in the groove portion.

Also, in the solid-state imaging device of the fifth embodiment (Example 5 of a solid-state imaging device) according to the present technology, the depth of the groove portion may be preferably not smaller than the thickness of at least one first rewiring line of the plurality of first rewiring lines.

With the solid-state imaging device of the fifth embodiment (Example 5 of a solid-state imaging device) according to the present technology, it is possible to increase the degree of freedom in the rewiring line layout or improve imaging characteristics, and further, both an increase in the degree of freedom in the rewiring line layout and improvement of imaging characteristics can be achieved. With the solid-state imaging device of the fifth embodiment (Example 5 of a solid-state imaging device) according to the present technology, the degree of freedom in rewiring design can be increased, and further, image noise can be reduced by the rewiring line layout in the stack structure.

In addition to the contents described above, the contents explained in the descriptions of the solid-state imaging devices of the first to fourth embodiments according to the present technology can be applied, without any change, to the solid-state imaging device of the fifth embodiment according to the present technology, unless there is some technical contradiction.

### 7. Sixth Embodiment (an Example of an Electronic Apparatus)

An electronic apparatus of a sixth embodiment according to the present technology is an electronic apparatus on which a solid-state imaging device of a first aspect according to the present technology is mounted, and the solid-state imaging device of the first aspect according to the present technology is a solid-state imaging device that includes: a semiconductor substrate that has a first principal surface on the light incident side and a second principal surface on the side opposite from the first principal surface, light receiving elements being arranged two-dimensionally on the first principal surface; a light transmissive substrate disposed above the light receiving elements; a wiring layer formed on the second principal surface of the semiconductor substrate; a first rewiring line electrically connected to an internal electrode formed in the wiring layer; and a second rewiring line formed on the side of the second principal surface of the semiconductor substrate.

Alternatively, an electronic apparatus of the sixth embodiment according to the present technology is an electronic apparatus on which a solid-state imaging device of a second aspect according to the present technology is mounted, and the solid-state imaging device of the second aspect according to the present technology is a solid-state imaging device that includes: a sensor substrate that includes a first semiconductor substrate having a first principal surface on the light incident side and a second principal surface on the side opposite from the first principal surface, light receiving elements being formed and arranged two-dimensionally on the first principal surface, and a first wiring layer formed on the second principal surface of the first semiconductor substrate; a circuit substrate that includes a second semiconductor substrate having a third principal surface on the light incident side and a fourth principal surface on the side opposite from the third principal surface, and a second wiring layer formed on the third principal surface of the second semiconductor substrate; a light transmissive substrate disposed above the light receiving elements; a first rewiring line electrically connected to an internal electrode formed in the second wiring layer; and a second rewiring line formed on the side of the fourth principal surface of the second semiconductor substrate. In this solid-state imaging device, the first wiring layer of the sensor substrate and the second wiring layer of the circuit substrate are bonded, to form a stack structure of the sensor substrate and the circuit substrate.

For example, an electronic apparatus of the sixth embodiment according to the present technology is an electronic apparatus in which a solid-state imaging device of one embodiment among the solid-state imaging devices of the first to fifth embodiments according to the present technology is mounted.

### 8. Examples of Use of Solid-State Imaging Devices to which the Present Technology is Applied

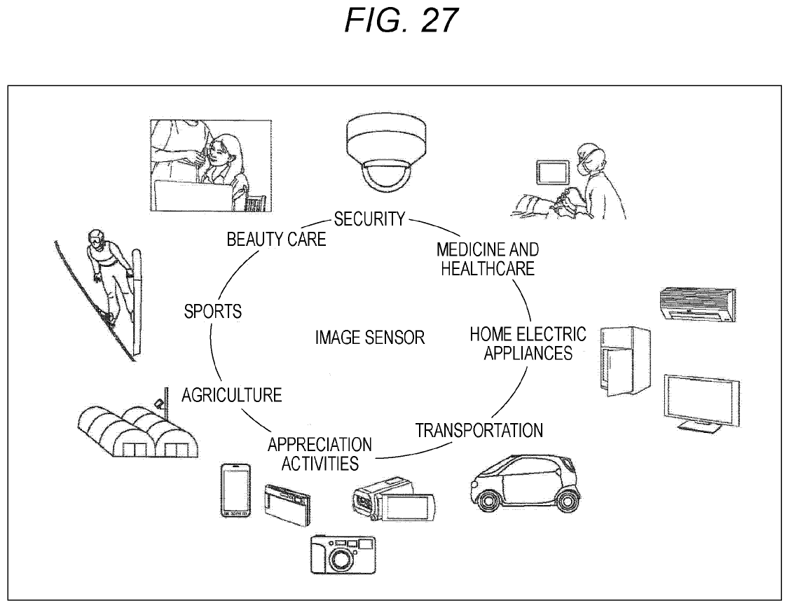

FIG. 27 is a diagram showing examples of use of solid-state imaging devices of the first to fifth embodiments according to the present technology as image sensors.

Solid-state imaging devices of the first to fifth embodiments described above can be used in various cases where light such as visible light, infrared light, ultraviolet light, or an X-ray is sensed, as described below, for example. That is, as shown in FIG. 27, solid-state imaging devices of any one of the first to fifth embodiments can be used in apparatuses (such as the electronic apparatus of the sixth embodiment described above, for example) that are used in the appreciation activity field where images are taken and are used in appreciation activities, the field of transportation, the field of home electric appliances, the fields of medicine and healthcare, the field of security, the field of beauty care, the field of sports, the field of agriculture, and the like, for example.

Specifically, in the appreciation activity field, a solid-state imaging device of any one of the first to fifth embodiments can be used in an apparatus for capturing images to be used in appreciation activities, such as a digital camera, a smartphone, or a portable telephone with a camera function, for example.

In the field of transportation, a solid-state imaging device of any one of the first to fifth embodiments can be used in an apparatus for transportation use, such as a vehicle-mounted sensor designed to capture images of the front, the back, the surroundings, the inside, and the like of an automobile, to perform safe driving such as an automatic stop and recognize the driver's condition or the like, a surveillance camera for monitoring running vehicles and roads, or a ranging sensor for measuring distances between vehicles or the like, for example.

In the field of home electric appliances, a solid-state imaging device of any one of the first to fifth embodiments can be used in an apparatus to be used as home electric appliances, such as a television set, a refrigerator, or an air conditioner, to capture images of gestures of users and operate the apparatus in accordance with the gestures, for example.

In the fields of medicine and healthcare, a solid-state imaging device of any one of the first to fifth embodiments can be used in an apparatus for medical use or healthcare use, such as an endoscope or an apparatus for receiving infrared light for angiography, for example.

In the field of security, a solid-state imaging device of any one of the first to fifth embodiments can be used in an apparatus for security use, such as a surveillance camera for crime prevention or a camera for personal authentication, for example.

In the field of beauty care, a solid-state imaging device of any one of the first to fifth embodiments can be used in an apparatus for beauty care use, such as a skin measurement apparatus designed to capture images of the skin or a microscope for capturing images of the scalp, for example.

In the field of sports, a solid-state imaging device of any one of the first to fifth embodiments can be used in an apparatus for sporting use, such as an action camera or a wearable camera for sports or the like, for example.

In the field of agriculture, a solid-state imaging device of any one of the first to fifth embodiments can be used in an apparatus for agricultural use, such as a camera for monitoring conditions of fields and crops, for example.

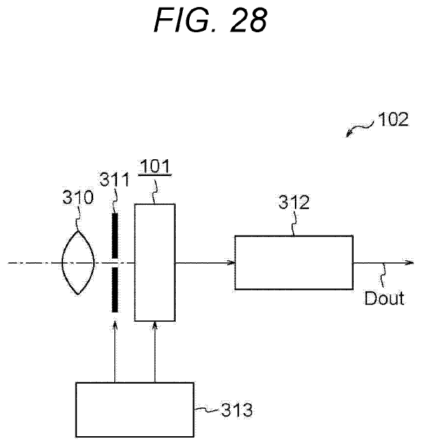

Next, examples of use of solid-state imaging devices of the first to fifth embodiments according to the present technology are specifically described. For example, a solid-state imaging device of any one of the first to fifth embodiments described above can be used as a solid-state imaging device 101 in an electronic apparatus of any type having an imaging function, such as a camera system like a digital still camera or a video camera, or a portable telephone having an imaging function. FIG. 28 shows a schematic configuration of an electronic apparatus 102 (a camera) as an example. This electronic apparatus 102 is a video camera capable of capturing a still image or a moving image, for example, and includes the solid-state imaging device 101, an optical system (an optical lens) 310, a shutter device 311, a drive unit 313 that drives the solid-state imaging device 101 and the shutter device 311, and a signal processing unit 312.

The optical system 310 guides image light (incident light) from the object to a pixel unit 101a of the solid-state imaging device 101. This optical system 310 may be formed with a plurality of optical lenses. The shutter device 311 controls the light irradiation period and the light blocking period for the solid-state imaging device 101. The drive unit 313 controls transfer operations of the solid-state imaging device 101 and shutter operations of the shutter device 311. The signal processing unit 312 performs various kinds of signal processing on a signal output from the solid-state imaging device 101. A video signal Dout subjected to the signal processing is stored into a storage medium such as a memory, or is output to a monitor or the like.

### 9. Example Application to an Endoscopic Surgery System

The present technology can be applied to various products. For example, the technology (the present technology) according to the present disclosure may be applied to an endoscopic surgery system.

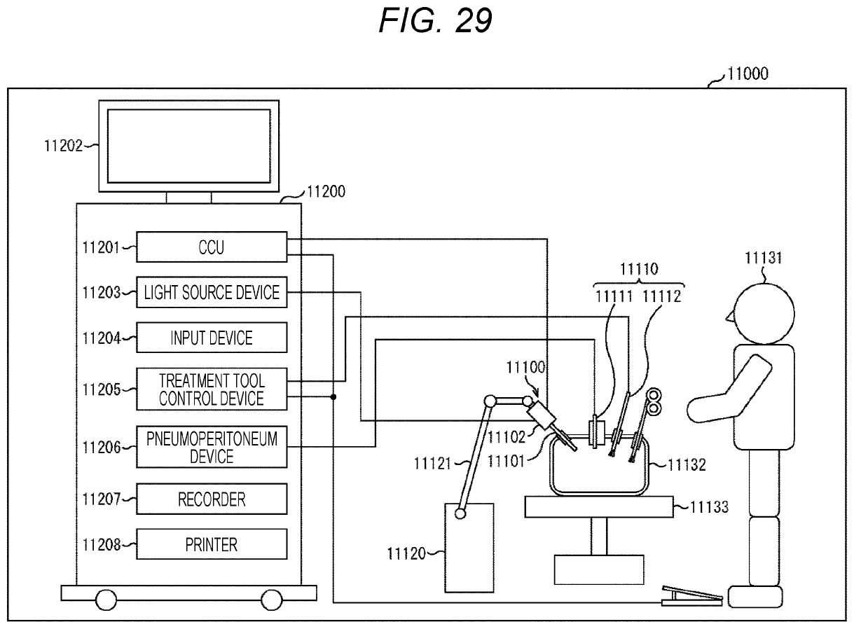

FIG. 29 is a diagram schematically showing an example configuration of an endoscopic surgery system to which the technology (the present technology) according to the present disclosure may be applied.

FIG. 29 shows a situation where a surgeon (a physician) 11131 is performing surgery on a patient 11132 on a patient bed 11133, using an endoscopic surgery system 11000. As shown in the drawing, the endoscopic surgery system 11000 includes an endoscope 11100, other surgical tools 11110 such as a pneumoperitoneum tube 11111 and an energy treatment tool 11112, a support arm device 11120 that supports the endoscope 11100, and a cart 11200 on which various kinds of devices for endoscopic surgery are mounted.

The endoscope 11100 includes a lens barrel 11101 that has a region of a predetermined length from the top end to be inserted into a body cavity of the patient 11132, and a camera head 11102 connected to the base end of the lens barrel 11101. In the example shown in the drawing, the endoscope 11100 is designed as a so-called rigid scope having a rigid lens barrel 11101. However, the endoscope 11100 may be designed as a so-called flexible scope having a flexible lens barrel.

At the top end of the lens barrel 11101, an opening into which an objective lens is inserted is provided. A light source device 11203 is connected to the endoscope 11100, and the light generated by the light source device 11203 is guided to the top end of the lens barrel by a light guide extending inside the lens barrel 11101, and is emitted toward the current observation target in the body cavity of the patient 11132 via the objective lens. Note that the endoscope 11100 may be a forward-viewing endoscope, an oblique-viewing endoscope, or a side-viewing endoscope.

An optical system and imaging elements are provided inside the camera head 11102, and reflected light (observation light) from the current observation target is converged on the imaging elements by the optical system. The observation light is photoelectrically converted by the imaging elements, and an electrical signal corresponding to the observation light, or an image signal corresponding to the observation image, is generated. The image signal is transmitted as RAW data to a camera control unit (CCU) 11201.

The CCU 11201 is formed with a central processing unit (CPU), a graphics processing unit (GPU), or the like, and collectively controls operations of the endoscope 11100 and a display device 11202. Further, the CCU 11201 receives an image signal from the camera head 11102, and subjects the image signal to various kinds of image processing, such as a development process (a demosaicing process), for example, to display an image based on the image signal.

Under the control of the CCU 11201, the display device 11202 displays an image based on the image signal subjected to the image processing by the CCU 11201.

The light source device 11203 is formed with a light source such as a light emitting diode (LED), for example, and supplies the endoscope 11100 with illuminating light for imaging the surgical site or the like.

An input device 11204 is an input interface to the endoscopic surgery system 11000. The user can input various kinds of information and instructions to the endoscopic surgery system 11000 via the input device 11204. For example, the user inputs an instruction or the like to change imaging conditions (such as the type of illuminating light, the magnification, and the focal length) for the endoscope 11100.

A treatment tool control device 11205 controls driving of the energy treatment tool 11112 for tissue cauterization, incision, blood vessel sealing, or the like. A pneumoperitoneum device 11206 injects a gas into a body cavity of the patient 11132 via the pneumoperitoneum tube 11111 to inflate the body cavity, for the purpose of securing the field of view of the endoscope 11100 and the working space of the surgeon. A recorder 11207 is a device capable of recording various kinds of information about the surgery. A printer 11208 is a device capable of printing various kinds of information relating to the surgery in various formats such as text, images, graphics, and the like.

Note that the light source device 11203 that supplies the endoscope 11100 with the illuminating light for imaging the surgical site can be formed with an LED, a laser light source, or a white light source that is a combination of an LED and a laser light source, for example. In a case where a white light source is formed with a combination of RGB laser light sources, the output intensity and the output timing of each color (each wavelength) can be controlled with high precision. Accordingly, the white balance of an image captured by the light source device 11203 can be adjusted. Alternatively, in this case, laser light from each of the RGB laser light sources may be emitted onto the current observation target in a time-division manner, and driving of the imaging elements of the camera head 11102 may be controlled in synchronization with the timing of the light emission. Thus, images corresponding to the respective RGB colors can be captured in a time-division manner. According to the method, a color image can be obtained without any color filter provided in the imaging elements.

Further, the driving of the light source device 11203 may also be controlled so that the intensity of light to be output is changed at predetermined time intervals. The driving of the imaging elements of the camera head 11102 is controlled in synchronism with the timing of the change in the intensity of the light, and images are acquired in a time-division manner and are then combined. Thus, a high dynamic range image with no black portions and no white spots can be generated.

Further, the light source device 11203 may also be designed to be capable of supplying light of a predetermined wavelength band compatible with special light observation. In special light observation, light of a narrower band than the illuminating light (or white light) at the time of normal observation is emitted, with the wavelength dependence of light absorption in body tissue being taken advantage of, for example. As a result, so-called narrow band light observation (narrow band imaging) is performed to image predetermined tissue such as a blood vessel in a mucosal surface layer or the like, with high contrast. Alternatively, in the special light observation, fluorescence observation for obtaining an image with fluorescence generated through emission of excitation light may be performed. In fluorescence observation, excitation light is emitted to body tissue so that the fluorescence from the body tissue can be observed (autofluorescence observation). Alternatively, a reagent such as indocyanine green (ICG) is locally injected into body tissue, and excitation light corresponding to the fluorescence wavelength of the reagent is emitted to the body tissue so that a fluorescent image can be obtained, for example. The light source device 11203 can be designed to be capable of supplying narrow band light and/or excitation light compatible with such special light observation.

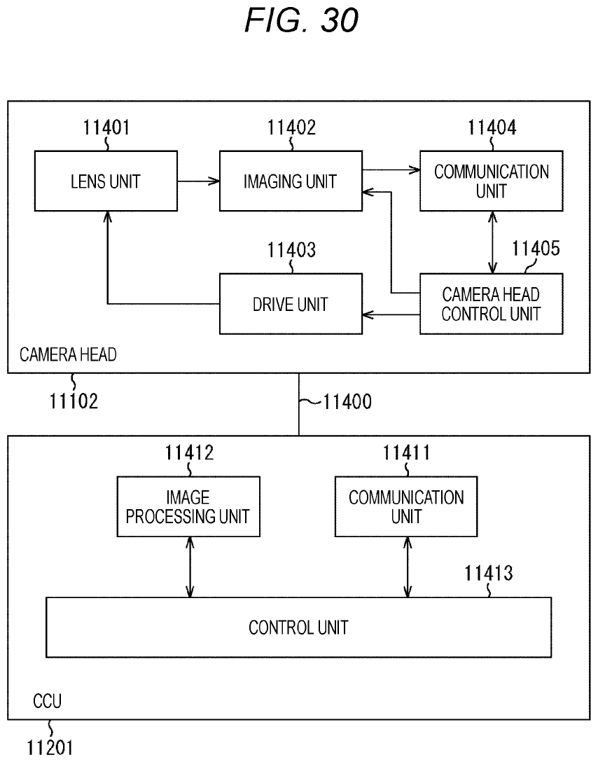

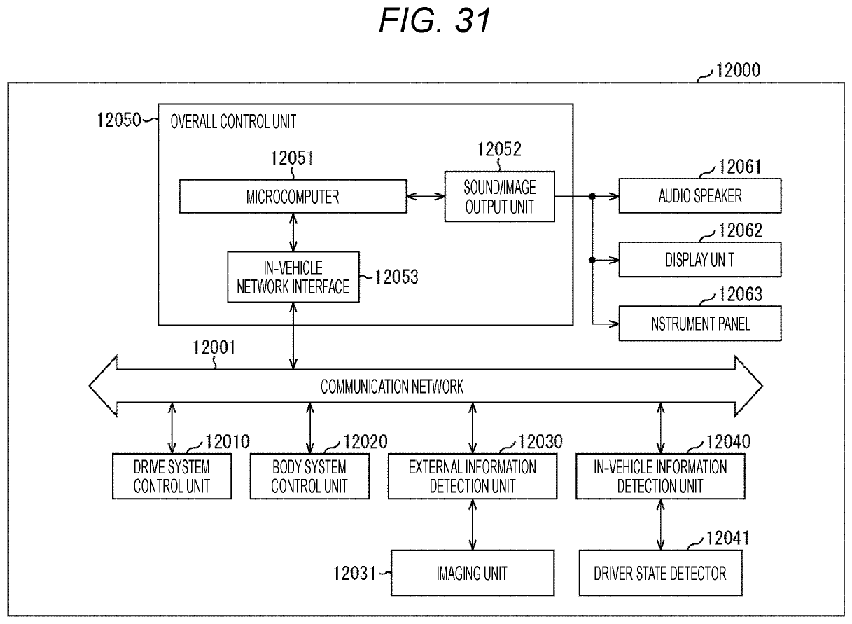

FIG. 30 is a block diagram showing an example of the functional configurations of the camera head 11102 and the CCU 11201 shown in FIG. 29.

The camera head 11102 includes a lens unit 11401, an imaging unit 11402, a drive unit 11403, a communication unit 11404, and a camera head control unit 11405. The CCU 11201 includes a communication unit 11411, an image processing unit 11412, and a control unit 11413. The camera head 11102 and the CCU 11201 are communicably connected to each other by a transmission cable 11400.

The lens unit 11401 is an optical system provided at the connecting portion with the lens barrel 11101. Observation light captured from the top end of the lens barrel 11101 is guided to the camera head 11102, and enters the lens unit 11401. The lens unit 11401 is formed with a combination of a plurality of lenses including a zoom lens and a focus lens.

The imaging unit 11402 is formed with imaging elements. The imaging unit 11402 may be formed with one imaging element (a so-called single-plate type), or may be formed with a plurality of imaging elements (a so-called multiple-plate type). In a case where the imaging unit 11402 is of a multiple-plate type, for example, image signals corresponding to the respective RGB colors may be generated by the respective imaging elements, and be then combined to obtain a color image. Alternatively, the imaging unit 11402 may be designed to include a pair of imaging elements for acquiring right-eye and left-eye image signals compatible with three-dimensional (3D) display. As the 3D display is conducted, the surgeon 11131 can grasp more accurately the depth of the body tissue at the surgical site. Note that, in a case where the imaging unit 11402 is of a multiple-plate type, a plurality of lens units 11401 is provided for the respective imaging elements.

Further, the imaging unit 11402 is not necessarily provided in the camera head 11102. For example, the imaging unit 11402 may be provided immediately behind the objective lens in the lens barrel 11101.

The drive unit 11403 is formed with an actuator, and, under the control of the camera head control unit 11405, moves the zoom lens and the focus lens of the lens unit 11401 by a predetermined distance along the optical axis. With this arrangement, the magnification and the focal point of the image captured by the imaging unit 11402 can be adjusted as appropriate.

The communication unit 11404 is formed with a communication device for transmitting and receiving various kinds of information to and from the CCU 11201. The communication unit 11404 transmits the image signal obtained as RAW data from the imaging unit 11402 to the CCU 11201 via the transmission cable 11400.

The communication unit 11404 also receives a control signal for controlling the driving of the camera head 11102 from the CCU 11201, and supplies the control signal to the camera head control unit 11405. The control signal includes information about imaging conditions, such as information for specifying the frame rate of captured images, information for specifying the exposure value at the time of imaging, and/or information for specifying the magnification and the focal point of captured images, for example.

Note that the above imaging conditions such as the frame rate, the exposure value, the magnification, and the focal point may be appropriately specified by the user, or may be automatically set by the control unit 11413 of the CCU 11201 on the basis of an acquired image signal. In the latter case, the endoscope 11100 has a so-called auto-exposure (AE) function, an auto-focus (AF) function, and an auto-white-balance (AWB) function.

The camera head control unit 11405 controls the driving of the camera head 11102, on the basis of a control signal received from the CCU 11201 via the communication unit 11404.

The communication unit 11411 is formed with a communication device for transmitting and receiving various kinds of information to and from the camera head 11102. The communication unit 11411 receives an image signal transmitted from the camera head 11102 via the transmission cable 11400.

Further, the communication unit 11411 also transmits a control signal for controlling the driving of the camera head 11102, to the camera head 11102. The image signal and the control signal can be transmitted through electrical communication, optical communication, or the like.

The image processing unit 11412 performs various kinds of image processing on an image signal that is RAW data transmitted from the camera head 11102.

The control unit 11413 performs various kinds of control relating to display of an image of the surgical portion or the like captured by the endoscope 11100, and a captured image obtained through imaging of the surgical site or the like.

For example, the control unit 11413 generates a control signal for controlling the driving of the camera head 11102.

Further, the control unit 11413 also causes the display device 11202 to display a captured image showing the surgical site or the like, on the basis of the image signal subjected to the image processing by the image processing unit 11412. In doing so, the control unit 11413 may recognize the respective objects shown in the captured image, using various image recognition techniques. For example, the control unit 11413 can detect the shape, the color, and the like of the edges of an object shown in the captured image, to recognize the surgical tool such as forceps, a specific body site, bleeding, the mist at the time of use of the energy treatment tool 11112, and the like. When causing the display device 11202 to display the captured image, the control unit 11413 may cause the display device 11202 to superimpose various kinds of surgery aid information on the image of the surgical site on the display, using the recognition result. As the surgery aid information is superimposed and displayed, and thus, is presented to the surgeon 11131, it becomes possible to reduce the burden on the surgeon 11131, and enable the surgeon 11131 to proceed with the surgery in a reliable manner.

The transmission cable 11400 connecting the camera head 11102 and the CCU 11201 is an electrical signal cable compatible with electric signal communication, an optical fiber compatible with optical communication, or a composite cable thereof.

Here, in the example shown in the drawing, communication is performed in a wired manner using the transmission cable 11400. However, communication between the camera head 11102 and the CCU 11201 may be performed in a wireless manner.

An example of an endoscopic surgery system to which the technique according to the present disclosure can be applied has been described above. The technology according to the present disclosure may be applied to the endoscope 11100, the imaging unit 11402 of the camera head 11102, and the like in the configuration described above, for example. Specifically, the solid-state imaging device 111 of the present disclosure can be applied to the imaging unit 10402. As the technology according to the present disclosure is applied to the endoscope 11100, (the imaging unit 11402 of) the camera head 11102, and the like, it is possible to improve the performance, the quality, and the like of the endoscope 11100, (the imaging unit 11402 of) the camera head 11102, and the like.

Although the endoscopic surgery system has been described as an example herein, the technology according to the present disclosure may be applied to a microscopic surgery system or the like, for example.

### 10. Example Applications to Mobile Structures

The technology (the present technology) according to the present disclosure can be applied to various products. For example, the technology according to the present disclosure may be embodied as a device mounted on any type of mobile structure, such as an automobile, an electrical vehicle, a hybrid electrical vehicle, a motorcycle, a bicycle, a personal mobility device, an airplane, a drone, a vessel, or a robot.

FIG. 31 is a block diagram schematically showing an example configuration of a vehicle control system that is an example of a mobile structure control system to which the technology according to the present disclosure may be applied.

A vehicle control system 12000 includes a plurality of electronic control units connected via a communication network 12001. In the example shown in FIG. 31, the vehicle control system 12000 includes a drive system control unit 12010, a body system control unit 12020, an external information detection unit 12030, an in-vehicle information detection unit 12040, and an overall control unit 12050. Further, a microcomputer 12051, a sound/image output unit 12052, and an in-vehicle network interface (I/F) 12053 are shown as the functional components of the overall control unit 12050.

The drive system control unit 12010 controls operations of the devices related to the drive system of the vehicle according to various programs. For example, the drive system control unit 12010 functions as control devices such as a driving force generation device for generating a driving force of the vehicle such as an internal combustion engine or a driving motor, a driving force transmission mechanism for transmitting the driving force to the wheels, a steering mechanism for adjusting the steering angle of the vehicle, and a braking device for generating a braking force of the vehicle.

The body system control unit 12020 controls operations of the various devices mounted on the vehicle body according to various programs. For example, the body system control unit 12020 functions as a keyless entry system, a smart key system, a power window device, or a control device for various lamps such as a headlamp, a backup lamp, a brake lamp, a turn signal lamp, a fog lamp, or the like. In this case, the body system control unit 12020 can receive radio waves transmitted from a portable device that substitutes for a key, or signals from various switches. The body system control unit 12020 receives inputs of these radio waves or signals, and controls the door lock device, the power window device, the lamps, and the like of the vehicle.

The external information detection unit 12030 detects information outside the vehicle equipped with the vehicle control system 12000. For example, an imaging unit 12031 is connected to the external information detection unit 12030. The external information detection unit 12030 causes the imaging unit 12031 to capture an image of the outside of the vehicle, and receives the captured image. On the basis of the received image, the external information detection unit 12030 may perform an object detection process for detecting a person, a vehicle, an obstacle, a sign, characters on the road surface, or the like, or perform a distance detection process.

The imaging unit 12031 is an optical sensor that receives light, and outputs an electrical signal corresponding to the amount of received light. The imaging unit 12031 can output an electrical signal as an image, or output an electrical signal as distance measurement information. Further, the light to be received by the imaging unit 12031 may be visible light, or may be invisible light such as infrared rays.

The in-vehicle information detection unit 12040 detects information about the inside of the vehicle. For example, a driver state detector 12041 that detects the state of the driver is connected to the in-vehicle information detection unit 12040. The driver state detector 12041 includes a camera that captures an image of the driver, for example, and, on the basis of detected information input from the driver state detector 12041, the in-vehicle information detection unit 12040 may calculate the degree of fatigue or the degree of concentration of the driver, or determine whether or not the driver is dozing off.

On the basis of the external/internal information acquired by the external information detection unit 12030 or the in-vehicle information detection unit 12040, the microcomputer 12051 can calculate the control target value of the driving force generation device, the steering mechanism, or the braking device, and output a control command to the drive system control unit 12010. For example, the microcomputer 12051 can perform cooperative control to achieve the functions of an advanced driver assistance system (ADAS), including vehicle collision avoidance or impact mitigation, follow-up running based on the distance between vehicles, vehicle velocity maintenance running, vehicle collision warning, vehicle lane deviation warning, or the like.

Further, the microcomputer 12051 can also perform cooperative control to conduct automatic driving or the like for autonomously running not depending on the operation of the driver, by controlling the driving force generation device, the steering mechanism, the braking device, or the like on the basis of information about the surroundings of the vehicle, the information having being acquired by the external information detection unit 12030 or the in-vehicle information detection unit 12040.

The microcomputer 12051 can also output a control command to the body system control unit 12020, on the basis of the external information acquired by the external information detection unit 12030. For example, the microcomputer 12051 controls the headlamp in accordance with the position of the leading vehicle or the oncoming vehicle detected by the external information detection unit 12030, and performs cooperative control to achieve an anti-glare effect by switching from a high beam to a low beam, or the like.

The sound/image output unit 12052 transmits an audio output signal and/or an image output signal to an output device that is capable of visually or audibly notifying the passenger(s) of the vehicle or the outside of the vehicle of information. In the example shown in FIG. 31, an audio speaker 12061, a display unit 12062, and an instrument panel 12063 are shown as output devices. The display unit 12062 may include an on-board display and/or a head-up display, for example.

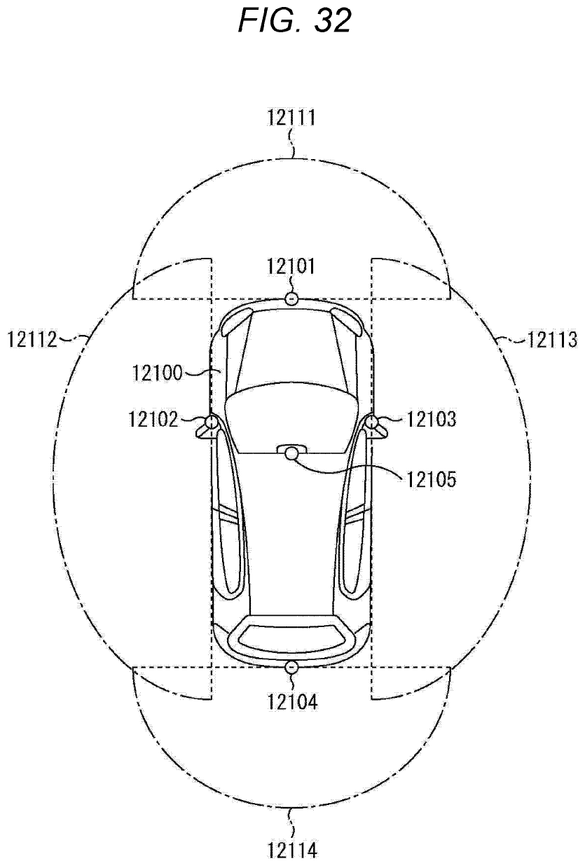

FIG. 32 is a diagram showing an example of installation positions of imaging units 12031.

In FIG. 32, a vehicle 12100 includes imaging units 12101, 12102, 12103, 12104, and 12105 as the imaging units 12031.

Imaging units 12101, 12102, 12103, 12104, and 12105 are provided at the following positions: the front end edge of a vehicle 12100, a side mirror, the rear bumper, a rear door, an upper portion of the front windshield inside the vehicle, and the like, for example. The imaging unit 12101 provided on the front end edge and the imaging unit 12105 provided on the upper portion of the front windshield inside the vehicle mainly capture images ahead of the vehicle 12100. The imaging units 12102 and 12103 provided on the side mirrors mainly capture images on the sides of the vehicle 12100. The imaging unit 12104 provided on the rear bumper or a rear door mainly captures images behind the vehicle 12100. The front images acquired by the imaging units 12101 and 12105 are mainly used for detection of a vehicle running in front of the vehicle 12100, a pedestrian, an obstacle, a traffic signal, a traffic sign, a lane, or the like.

Note that FIG. 32 shows an example of the imaging ranges of the imaging units 12101 to 12104. An imaging range 12111 indicates the imaging range of the imaging unit 12101 provided on the front end edge, imaging ranges 12112 and 12113 indicate the imaging ranges of the imaging units 12102 and 12103 provided on the respective side mirrors, and an imaging range 12114 indicates the imaging range of the imaging unit 12104 provided on the rear bumper or a rear door. For example, image data captured by the imaging units 12101 to 12104 are superimposed on one another, so that an overhead image of the vehicle 12100 viewed from above is obtained.

At least one of the imaging units 12101 to 12104 may have a function of acquiring distance information. For example, at least one of the imaging units 12101 to 12104 may be a stereo camera including a plurality of imaging elements, or may be imaging elements having pixels for phase difference detection.

For example, on the basis of distance information obtained from the imaging units 12101 to 12104, the microcomputer 12051 calculates the distances to the respective three-dimensional objects within the imaging ranges 12111 to 12114, and temporal changes in the distances (the velocities relative to the vehicle 12100). In this manner, the three-dimensional object that is the closest three-dimensional object on the traveling path of the vehicle 12100 and is traveling at a predetermined velocity (0 km/h or higher, for example) in substantially the same direction as the vehicle 12100 can be extracted as the vehicle running in front of the vehicle 12100. Further, the microcomputer 12051 can set beforehand an inter-vehicle distance to be maintained in front of the vehicle running in front of the vehicle 12100, and can perform automatic brake control (including follow-up stop control), automatic acceleration control (including follow-up start control), and the like. In this manner, it is possible to perform cooperative control to conduct automatic driving or the like to autonomously travel not depending on the operation of the driver.

For example, in accordance with the distance information obtained from the imaging units 12101 to 12104, the microcomputer 12051 can extract three-dimensional object data concerning three-dimensional objects under the categories of two-wheeled vehicles, regular vehicles, large vehicles, pedestrians, utility poles, and the like, and use the three-dimensional object data in automatically avoiding obstacles. For example, the microcomputer 12051 classifies the obstacles in the vicinity of the vehicle 12100 into obstacles visible to the driver of the vehicle 12100 and obstacles difficult to visually recognize. The microcomputer 12051 then determines collision risks indicating the risks of collision with the respective obstacles. If a collision risk is equal to or higher than a set value, and there is a possibility of collision, the microcomputer 12051 can output a warning to the driver via the audio speaker 12061 and the display unit 12062, or can perform driving support for avoiding collision by performing forced deceleration or avoiding steering via the drive system control unit 12010.

At least one of the imaging units 12101 to 12104 may be an infrared camera that detects infrared rays. For example, the microcomputer 12051 can recognize a pedestrian by determining whether or not a pedestrian exists in images captured by the imaging units 12101 to 12104. Such pedestrian recognition is carried out through a process of extracting feature points from the images captured by the imaging units 12101 to 12104 serving as infrared cameras, and a process of performing a pattern matching on the series of feature points indicating the outlines of objects and determining whether or not there is a pedestrian, for example. If the microcomputer 12051 determines that a pedestrian exists in the images captured by the imaging units 12101 to 12104, and recognizes a pedestrian, the sound/image output unit 12052 controls the display unit 12062 to display a rectangular contour line for emphasizing the recognized pedestrian in a superimposed manner. Further, the sound/image output unit 12052 may also control the display unit 12062 to display an icon or the like indicating the pedestrian at a desired position.

An example of a vehicle control system to which the technology (the present technology) according to the present disclosure may be applied has been described above. The technology according to the present disclosure may be applied to the imaging units 12031 and the like among the components described above, for example. Specifically, the solid-state imaging device 111 of the present disclosure can be applied to the imaging units 12031. As the technique according to the present disclosure is applied to the imaging units 12031, it is possible to improve the performance, the quality, and the like of the imaging units 12031.

Note that the present technology is not limited to the embodiments and example applications described above, and various modifications may be made to them without departing from the scope of the present technology.

Further, the advantageous effects described in this specification are merely examples, and the advantageous effects of the present technology are not limited to them and may include other effects.

The present technology may also be embodied in the configurations described below.

[1]

A solid-state imaging device including:

a semiconductor substrate that has a first principal surface on a light incident side and a second principal surface on a side opposite from the first principal surface, a light receiving element being disposed two-dimensionally on the first principal surface;

a light transmissive substrate disposed above the light receiving element;

a wiring layer formed on the second principal surface of the semiconductor substrate;

a first rewiring line electrically connected to an internal electrode formed in the wiring layer; and

a second rewiring line formed on a side of the second principal surface of the semiconductor substrate.

[2]

The solid-state imaging device according to [1], in which the second rewiring line is disposed below the first rewiring line.

[3]

The solid-state imaging device according to [1] or [2] claim 1, in which

the first rewiring line is formed with a plurality of first rewiring lines,

the second rewiring line is formed with a plurality of second rewiring lines, and

at least one first rewiring line of the plurality of first rewiring lines and at least one second rewiring line of the plurality of second rewiring lines are connected.

[4]

The solid-state imaging device according to any one of [1] to [3], in which

the first rewiring line is formed with a plurality of first rewiring lines,

the second rewiring line is formed with a plurality of second rewiring lines,

the wiring layer includes a plurality of signal lines formed in a predetermined direction, and

the plurality of first rewiring lines and the plurality of second rewiring lines are arranged to generate a plurality of magnetic fields having different magnetic directions from one another in a region between two adjacent signal lines of the plurality of signal lines.

[5]

The solid-state imaging device according to [4], in which

at least one pair of one first rewiring line of the plurality of first rewiring lines and one second rewiring line of the plurality of second rewiring lines is formed in a vertical direction, and,

in the one pair, a direction of a first current flowing in the one first rewiring line and a direction of a second current flowing in the one second rewiring line are the opposite from each other.

[6]

The solid-state imaging device according to any one of [1] to [5], in which

the first rewiring line is formed with a plurality of first rewiring lines,

the second rewiring line is formed with a plurality of second rewiring lines,

the wiring layer includes a plurality of signal lines formed in a predetermined direction, and

at least one first rewiring line of the plurality of first rewiring lines and/or at least one second rewiring line of the plurality of second rewiring lines covers at least part of at least one signal line of the plurality of signal lines, when viewed from a side opposite from the light incident side.

[7]

The solid-state imaging device according to any one of [1] to [6], in which

the first rewiring line is formed with a plurality of first rewiring lines,

a groove portion is formed on a side of the second principal surface of the semiconductor substrate, and

at least part of at least one first rewiring line of the plurality of first rewiring lines is formed in the groove portion.

[8]

The solid-state imaging device according to [7], in which a depth of the groove portion is not smaller than a thickness of at least one first rewiring line of the plurality of first rewiring lines.

[9]

A solid-state imaging device including:

a sensor substrate that includes:

a first semiconductor substrate having a first principal surface on a light incident side and a second principal surface on a side opposite from the first principal surface, a light receiving element being disposed two-dimensionally on the first principal surface; and

a first wiring layer formed on the second principal surface of the first semiconductor substrate;

a circuit substrate that includes:

a second semiconductor substrate having a third principal surface on the light incident side and a fourth principal surface on a side opposite from the third principal surface; and

a second wiring layer formed on the third principal surface of the second semiconductor substrate;

a light transmissive substrate disposed above the light receiving element;

a first rewiring line electrically connected to an internal electrode formed in the second wiring layer; and

a second rewiring line formed on a side of the fourth principal surface of the second semiconductor substrate,

in which the first wiring layer of the sensor substrate and the second wiring layer of the circuit substrate are bonded, to form a stack structure of the sensor substrate and the circuit substrate.

[10]

The solid-state imaging device according to [9], in which the second rewiring line is disposed below the first rewiring line.

[11]

The solid-state imaging device according to [9] or [10], in which

the first rewiring line is formed with a plurality of first rewiring lines,

the second rewiring line is formed with a plurality of second rewiring lines, and

at least one first rewiring line of the plurality of first rewiring lines and at least one second rewiring line of the plurality of second rewiring lines are connected.

[12]

The solid-state imaging device according to any one of [9] to [11], in which

the first rewiring line is formed with a plurality of first rewiring lines,

the second rewiring line is formed with a plurality of second rewiring lines,

the second wiring layer includes a plurality of signal lines formed in a predetermined direction, and

the plurality of first rewiring lines and the plurality of second rewiring lines are arranged to generate a plurality of magnetic fields having different magnetic directions from one another in a region between two adjacent signal lines of the plurality of signal lines.

[13]

The solid-state imaging device according to [12], in which

at least one pair of one first rewiring line of the plurality of first rewiring lines and one second rewiring line of the plurality of second rewiring lines is formed in a vertical direction, and

in the one pair, a direction of a first current flowing in the one first rewiring line and a direction of a second current flowing in the one second rewiring line are the opposite from each other.

[14]

The solid-state imaging device according to any one of [9] to [13], in which

the first rewiring line is formed with a plurality of first rewiring lines,

the second rewiring line is formed with a plurality of second rewiring lines,

the wiring layer includes a plurality of signal lines formed in a predetermined direction, and

at least one first rewiring line of the plurality of first rewiring lines and/or at least one second rewiring line of the plurality of second rewiring lines covers at least part of at least one signal line of the plurality of signal lines, when viewed from a side opposite from the light incident side.

[15]

The solid-state imaging device according to any one of [9] to [14], in which

the first rewiring line is formed with a plurality of first rewiring lines,

a groove portion is formed on a side of the fourth principal surface of the second semiconductor substrate, and

at least part of at least one first rewiring line of the plurality of first rewiring lines is formed in the groove portion.

[16]

The solid-state imaging device according to [15], in which a depth of the groove portion is not smaller than a thickness of at least one first rewiring line of the plurality of first rewiring lines.

[17]

An electronic apparatus including the solid-state imaging device according to any one of [1] to [16].

### REFERENCE SIGNS LIST

1, 3, 4 Solid-state imaging device
108, 208, 408, 508, 608 Internal electrode
110, 210, 310, 410, 510, 610 First rewiring line
112, 212, 312, 412, 512 Second rewiring line

1. A solid-state imaging device comprising:
a semiconductor substrate that has a first principal surface on a light incident side and a second principal surface on a side opposite from the first principal surface, a light receiving element being disposed two-dimensionally on the first principal surface;
a light transmissive substrate disposed above the light receiving element;
a wiring layer formed on the second principal surface of the semiconductor substrate;
a first rewiring line electrically connected to an internal electrode formed in the wiring layer; and
a second rewiring line formed on a side of the second principal surface of the semiconductor substrate.

2. The solid-state imaging device according to claim 1, wherein the second rewiring line is disposed below the first rewiring line.

3. The solid-state imaging device according to claim 1, wherein
the first rewiring line is formed with a plurality of first rewiring lines,
the second rewiring line is formed with a plurality of second rewiring lines, and
at least one first rewiring line of the plurality of first rewiring lines and at least one second rewiring line of the plurality of second rewiring lines are connected.

4. The solid-state imaging device according to claim 1, wherein
the first rewiring line is formed with a plurality of first rewiring lines,
the second rewiring line is formed with a plurality of second rewiring lines,
the wiring layer includes a plurality of signal lines formed in a predetermined direction, and
the plurality of first rewiring lines and the plurality of second rewiring lines are arranged to generate a plurality of magnetic fields having different magnetic directions from one another in a region between two adjacent signal lines of the plurality of signal lines.

5. The solid-state imaging device according to claim 4, wherein
at least one pair of one first rewiring line of the plurality of first rewiring lines and one second rewiring line of the plurality of second rewiring lines is formed in a vertical direction, and,
in the one pair, a direction of a first current flowing in the one first rewiring line and a direction of a second current flowing in the one second rewiring line are the opposite from each other.

6. The solid-state imaging device according to claim 1, wherein
the first rewiring line is formed with a plurality of first rewiring lines,
the second rewiring line is formed with a plurality of second rewiring lines,
the wiring layer includes a plurality of signal lines formed in a predetermined direction, and
at least one first rewiring line of the plurality of first rewiring lines and/or at least one second rewiring line of the plurality of second rewiring lines covers at least part of at least one signal line of the plurality of signal lines, when viewed from a side opposite from the light incident side.

7. The solid-state imaging device according to claim 1, wherein
the first rewiring line is formed with a plurality of first rewiring lines,
a groove portion is formed on a side of the second principal surface of the semiconductor substrate, and
at least part of at least one first rewiring line of the plurality of first rewiring lines is formed in the groove portion.

8. The solid-state imaging device according to claim 7, wherein a depth of the groove portion is not smaller than a thickness of at least one first rewiring line of the plurality of first rewiring lines.

9. A solid-state imaging device comprising:
a sensor substrate that includes:
a first semiconductor substrate having a first principal surface on a light incident side and a second principal surface on a side opposite from the first principal surface, a light receiving element being disposed two-dimensionally on the first principal surface; and
a first wiring layer formed on the second principal surface of the first semiconductor substrate;
a circuit substrate that includes:
a second semiconductor substrate having a third principal surface on the light incident side and a fourth principal surface on a side opposite from the third principal surface; and
a second wiring layer formed on the third principal surface of the second semiconductor substrate;
a light transmissive substrate disposed above the light receiving element;
a first rewiring line electrically connected to an internal electrode formed in the second wiring layer; and
a second rewiring line formed on a side of the fourth principal surface of the second semiconductor substrate,
wherein the first wiring layer of the sensor substrate and the second wiring layer of the circuit substrate are bonded, to form a stack structure of the sensor substrate and the circuit substrate.

10. The solid-state imaging device according to claim 9, wherein the second rewiring line is disposed below the first rewiring line.

11. The solid-state imaging device according to claim 9, wherein
the first rewiring line is formed with a plurality of first rewiring lines,
the second rewiring line is formed with a plurality of second rewiring lines, and
at least one first rewiring line of the plurality of first rewiring lines and at least one second rewiring line of the plurality of second rewiring lines are connected.

12. The solid-state imaging device according to claim 9, wherein
the first rewiring line is formed with a plurality of first rewiring lines,
the second rewiring line is formed with a plurality of second rewiring lines,
the second wiring layer includes a plurality of signal lines formed in a predetermined direction, and
the plurality of first rewiring lines and the plurality of second rewiring lines are arranged to generate a plurality of magnetic fields having different magnetic directions from one another in a region between two adjacent signal lines of the plurality of signal lines.

13. The solid-state imaging device according to claim 12, wherein
at least one pair of one first rewiring line of the plurality of first rewiring lines and one second rewiring line of the plurality of second rewiring lines is formed in a vertical direction, and,
in the one pair, a direction of a first current flowing in the one first rewiring line and a direction of a second current flowing in the one second rewiring line are the opposite from each other.

14. The solid-state imaging device according to claim 9, wherein
the first rewiring line is formed with a plurality of first rewiring lines,
the second rewiring line is formed with a plurality of second rewiring lines,
the wiring layer includes a plurality of signal lines formed in a predetermined direction, and
at least one first rewiring line of the plurality of first rewiring lines and/or at least one second rewiring line of the plurality of second rewiring lines covers at least part of at least one signal line of the plurality of signal lines, when viewed from a side opposite from the light incident side.

15. The solid-state imaging device according to claim 9, wherein
the first rewiring line is formed with a plurality of first rewiring lines,
a groove portion is formed on a side of the fourth principal surface of the second semiconductor substrate, and
at least part of at least one first rewiring line of the plurality of first rewiring lines is formed in the groove portion.

16. The solid-state imaging device according to claim 15, wherein a depth of the groove portion is not smaller than a thickness of at least one first rewiring line of the plurality of first rewiring lines.

17. An electronic apparatus comprising the solid-state imaging device according to claim 1.

18. An electronic apparatus comprising the solid-state imaging device according to claim 9.
